(19) United States
(12) Patent Application Publication
(10) Pub. No.: US 2022/0101980 A1
(43) Pub. Date: Mar. 31, 2022

(54) System, Method, and Computer Program Product for Vascular Access Device Placement

(71) Applicant: Becton, Dickinson and Company, Franklin Lakes, NJ (US)

(72) Inventors: Ashley Rachel Rothenberg, Morris Plains, NJ (US); Erik Kurt Witt, Oakland, NJ (US); Mark Jesser, Wyckoff, NJ (US); Ami Kumordzie, New York, NY (US)

(21) Appl. No.: 17/488,981

(22) Filed: Sep. 29, 2021

Related U.S. Application Data
(60) Provisional application No. 63/085,294, filed on Sep. 30, 2020.

(51) Int. Cl. G16H 20/40; G16H 40/20; G16H 10/60; G16H 50/20

(52) U.S. Cl. CPC G16H 20/40; G16H 40/20; A61M 39/02; G16H 50/20; G16H 10/60

(57) ABSTRACT

Provided is a method for vascular access device placement. The method may include receiving clinician data associated with at least one clinician including a first clinician. Patient data associated with at least one patient including a first patient may be received. At least one first probability of successful placement of at least one vascular access device by the first clinician at at least one access point of the first patient may be generated based on the patient data associated with the first patient and the clinician data associated with the first clinician. An instruction message for placement of the at least one vascular access device may be communicated based on the at least one first probability to a user device. A system and computer program product are also disclosed.

### CROSS-REFERENCE TO RELATED APPLICATION

The present application claims priority to U.S. Provisional Application Ser. No. 63/085,294, entitled “System, Method, and Computer Program Product for Vascular Access Device Placement”, filed Sep. 30, 2020, the entire disclosure of which is incorporated by reference in its entirety.

### BACKGROUND

### 1. Field

This disclosed subject matter relates generally to systems, methods, and products for vascular access device placement and, in some particular embodiments or aspects, to a system, method, and computer program product for placement of a vascular access device (e.g., catheter and/or the like) based on clinician data and patient data.

### 2. Technical Considerations

Certain patients have a need for placement of a vascular access device (e.g., intravenous (IV) line, catheter, needle, cannula, and/or the like). Depending on characteristics of the patient, the type and size of vascular access device used, and the access point (e.g., target vein) at which the vascular access device will be placed, certain placements of vascular access devices may be more difficult than others. Additionally, certain clinicians may be more likely to succeed at placements of vascular access devices (especially the more difficult placements thereof) than other clinicians, e.g., based on level of experience, training, etc.

Allowing clinicians with less experience and/or training the opportunity to attempt placements of vascular access devices is important for the ongoing development of such clinicians (e.g., increasing their level of experience and/or training). However, unsuccessful attempts to place vascular access devices may result in pain for the patient, dissatisfaction of the patient, fear of vascular access devices in the patient, wasted resources (e.g., needing to dispose of the vascular access devices, disinfecting products, bandages, and/or the like), and wasted time. To improve chances of successful placement of vascular access devices, a different clinician (e.g., one with more experience and/or training) may be assigned to attempt the placement of such vascular access devices or other interventions, such as a guidance system, may be used by a clinician to guide the placement of the vascular access device. However, assigning another physician or using another intervention, such as a guidance system, may result in inefficient use of resources (e.g., if only a finite number of clinicians and/or guidance systems are available in a setting, the additional clinician or the guidance system may be unavailable to help other patients) and increased cost (e.g., hiring additional clinicians or purchasing additional guidance systems may increase costs per patient). Moreover, assigning another physician may deprive the initially assigned clinician from an opportunity for learning (e.g., increasing the initial clinician's experience and/or training) by attempting to place the vascular access device.

Additionally, if multiple access points are available for placement of the vascular access device and/or multiple types of vascular access devices are available, the clinician may not know which of the multiple access points and/or which of the multiple types of vascular access devices to use for the particular patient at that particular time.

### SUMMARY

Accordingly, it is an object of the presently disclosed subject matter to provide systems, methods, and products for vascular access device placement.

According to non-limiting embodiments or aspects, provided is a method for vascular access device placement. In some non-limiting embodiments or aspects, a method for vascular access device placement may include receiving clinician data associated with at least one clinician, and the at least one clinician may include a first clinician. Patient data associated with at least one patient may be received, and the at least one patient may include a first patient. At least one first probability of successful placement of at least one vascular access device by the first clinician at at least one access point of the first patient may be generated based on the patient data associated with the first patient and the clinician data associated with the first clinician. An instruction message for placement of the at least one vascular access device may be communicated based on the at least one first probability to a user device.

In some non-limiting embodiments or aspects, the clinician data may include at least one of years of experience, handedness, level of training, years of degree, whether the clinician is a member of an intravenous (IV) team, salary, a number of attempted placements of vascular access devices, a number of successful placements of the vascular access devices, a number of attempts per successful placement of each vascular access device, a number of attempted placements of the vascular access devices per patient, a vascular access device placement success ratio, an access point for each attempted placement of the vascular access devices, a number of successful placements of the vascular access devices per selectable subperiod over a selectable time period, whether at least one guidance system was used for each attempted placement of the vascular access devices, whether the at least one guidance system was used for each successful placement of the vascular access devices, any combination thereof, and/or the like.

In some non-limiting embodiments or aspects, the patient data may include at least one of height, weight, body mass index (BMI), sex, handedness, dominant side, current diagnostic code, one or more comorbidity diagnostic codes, previous intravenous (IV) treatments, skin pallor, skin shade, whether the patient has a tremor or other involuntary movement disorder, whether the patient has a condition requiring restraint or sedation, a categorization as Difficult Intra Venous Access (DIVA) or Adult Difficult Intra Venous Access (A-DIVA), an American Society of Anesthesiologists (ASA) physical status classification, whether the patient has an unplanned indication for surgery, whether the patient has undergone preoperative fasting, palpability of a target vein at the at least one access point, visibility of a target vein at the at least one access point, a known history of difficult peripheral IV cannulation, a dilated vein diameter smaller than a selectable threshold, size of the vascular access device, at least one available access point on at least one extremity of the patient, whether the patient has at least one vascular disease, whether the patient has preoperative hypovolemia, whether the patient has renal insufficiency, a level of fear of needles of the patient, a measure of difficulty of maintaining peripheral IV access once a respective vascular access devices is placed, a number of attempted placements of vascular access devices for the patient, a number of successful placements of the vascular access devices for the patient, a number of attempts per successful placement of each vascular access device for the patient, a vascular access device placement success ratio for the patient, an access point for each attempted placement of the vascular access devices for the patient, any combination thereof, and/or the like.

In some non-limiting embodiments or aspects, generating the at least one first probability may include generating the at least one first probability using a neural network model. Additionally or alternatively, the neural network model may include at least one neural network trained based on historical clinician data associated with a plurality of clinicians and historical patient data associated with a plurality of patients. In some non-limiting embodiments or aspects, before generating the at least one first probability, the at least one neural network of the neural network model may be trained based on the historical clinician data and the historical patient data.

In some non-limiting embodiments or aspects, first patient access point data associated with the at least one access point of the first patient may be received (e.g., as part of the patient data, separately from the patient data, and/or the like). Additionally or alternatively, generating the at least one first probability may include generating the at least one first probability based on the patient data associated with the first patient, the clinician data associated with the first clinician, and the first patient access point data.

In some non-limiting embodiments or aspects, the at least one clinician may include a plurality of clinicians (including the first clinician). Additionally or alternatively, the at least one patient may include a plurality of patients (including the first patient). In some non-limiting embodiments or aspects, first clinician identification data associated with the first clinician of the plurality of clinicians may be received. Additionally or alternatively, first patient identification data associated with the first patient of the plurality of patients may be received.

In some non-limiting embodiments or aspects, guideline data associated with at least one recommended access point for each of at least one vascular access procedure may be received. Additionally or alternatively, vascular access device data associated with the at least one vascular access device and/or cost data associated with supplies required for an attempted placement of the at least one vascular access device may be received. In some non-limiting embodiments or aspects, the instruction message indicating an instruction to place a first vascular access device of the at least one vascular access device at a first access point of the at least one access point may be generated based on the at least one first probability, the guideline data, the vascular access device data, and the cost data.

In some non-limiting embodiments or aspects, before communicating the instruction message, whether the at least one first probability satisfies a threshold may be determined. Additionally or alternatively, the instruction message indicating an instruction for the first clinician to place the at least one vascular access device at a first access point of the at least one access point may be generated based on the at least one first probability satisfying the threshold.

In some non-limiting embodiments or aspects, before communicating the instruction message, the first probability may be determined to not satisfy a threshold. Additionally or alternatively, the instruction message indicating an instruction for at least one intervention for placement of the at least one vascular access device may be generated based on the at least one first probability failing to satisfy the threshold. In some non-limiting embodiments or aspects, the at least one intervention may be at least one of a second clinician for placement of the vascular access device, at least one guidance system for placement of the vascular access device, any combination thereof, and/or the like.

In some non-limiting embodiments or aspects, before generating the instruction message, at least one of the following may be generated: at least one second probability of successful placement of the at least one vascular access device by the first clinician at the at least one access point of the first patient based on the patient data associated with the first patient, the clinician data associated with the first clinician, and guidance data associated with at least one guidance system; at least one third probability of successful placement of the at least one vascular access device by a second clinician of the at least one clinician at the at least one access point of the first patient based on the patient data associated with the first patient and the clinician data associated with the second clinician; or at least one fourth probability of successful placement of the at least one vascular access device by the second clinician at the at least one access point of the first patient based on the patient data associated with the first patient, the clinician data associated with the second clinician, and the guidance data associated with at least one guidance system.

In some non-limiting embodiments or aspects, at least one first predicted patient satisfaction for an attempted placement of the at least one vascular access device by the first clinician at the at least one access point of the first patient may be generated based on the patient data associated with the first patient and the clinician data associated with the first clinician. Additionally or alternatively, the instruction message for placement of the at least one vascular access device may be generated based on the at least one first probability and the at least one first predicted patient satisfaction. In some non-limiting embodiments or aspects, communicating the instruction message may include communicating the instruction message for placement of the at least one vascular access device based on the at least one first probability and the at least one first predicted patient satisfaction to the user device.

In some non-limiting embodiments or aspects, input data may be received based on an attempted placement of the at least one vascular access device by the first clinician at the at least one access point of the first patient. Additionally or alternatively, the input data may be at least one of: an update to the patient data associated with the first patient, an update to the clinician data associated with the first clinician, a side of the patient in which the attempted placement of the at least one vascular access device was performed, a first access point of the at least one access point at which the attempted placement of the at least one vascular access device was performed, a visibility of a target vein at the at least one access point, a dilated vein diameter of the target vein at the at least one access point, a palpability of the target vein at the at least one access point, a size of the at least one vascular access device, a type of the at least one vascular access device, a number of attempted placements of the at least one vascular access device, an indication of whether the attempted placement of the at least one vascular access device was successful, a time from when placement of the at least one vascular access device was ordered to the attempted placement of the at least one vascular access device, an indication of whether at least one intervention for placement of the at least one vascular access device was used, a level of fear of the attempted placement of the at least one vascular access device of the patient, a first pain score of the patient during the attempted placement of the at least one vascular access device, a second pain score of the patient after the attempted placement of the at least one vascular access device, a patient satisfaction score for the attempted placement of the at least one vascular access device, any combination thereof, and/or the like.

In some non-limiting embodiments or aspects, at least one of the following may be generated: at least one predicted cost for placement of the at least one vascular access device, at least one expected time from when placement of the at least one vascular access device was ordered to the successful placement of the at least one vascular access device, at least one first expected pain score of the patient during placement of the at least one vascular access device, at least one second expected pain score of the patient after placement of the at least one vascular access device, at least one difficulty score for placement of the at least one vascular access device at the at least one access point of the first patient, any combination thereof, and/or the like.

In some non-limiting embodiments or aspects, at least one graphical user interface (GUI) may be generated to indicate at least one of: an average time from when placement of the at least one vascular access device was ordered to the successful placement of the at least one vascular access device as a function of patient difficulty, a vascular access device placement success ratio as a function of the patient difficulty, a first average pain score during placement of the at least one vascular access device as a function of patient difficulty, a second average pain score after placement of the at least one vascular access device as a function of patient difficulty, an average patient satisfaction score, a summary of placements of vascular access devices, any combination thereof, and/or the like.

According to non-limiting embodiments or aspects, provided is a system for vascular access device placement. In some non-limiting embodiments or aspects, a system for vascular access device placement may include at least one clinician identification device, and the at least one clinician identification device may include a first clinician identification device associated with a first clinician. At least one patient identification device may include a first patient identification device associated with a first patient. At least one vascular access device may include a vascular access device identification device associated therewith. At least one reader device may be configured to communicate with the at least one clinician identification device, the at least one patient identification device, and each vascular access device identification device. The reader may be connected to a communication network. At least one user device may be connected to the communication network. At least one database may be connected to the communication network. The at least one database may store clinician data associated with a plurality of clinicians, and the plurality of clinicians may include the first clinician. The at least one database may store patient data associated with a plurality of patients, and the plurality of patients may include the first patient. The patient data for the first patient may include access point data associated with at least one access point of the first patient. The at least one database may store vascular access device data associated with a plurality of vascular access devices, and the plurality of vascular access devices may include the at least one vascular access device. A vascular access device placement modeling system may be connected to the communication network. The vascular access device placement modeling system may receive first clinician identification data associated with the first clinician from the first clinician identification device via the reader device. The vascular access device placement modeling system may receive first patient identification data associated with the first patient from the first patient identification device via the reader device. The vascular access device placement modeling system may receive vascular access device identification data associated with the at least one vascular access device from the vascular access device identification device associated with each of the at least one vascular access device via the reader device. The vascular access device placement modeling system may retrieve the clinician data associated with the first clinician from the at least one database. The vascular access device placement modeling system may retrieve the patient data associated with the first patient from the at least one database. The vascular access device placement modeling system may retrieve the vascular access device data associated with each of the at least one vascular access device from the at least one database. The vascular access device placement modeling system may generate at least one first probability of successful placement of the at least one vascular access device by the first clinician at the at least one access point of the first patient based on the patient data associated with the first patient, the clinician data associated with the first clinician, and the vascular access device data associated with each of the at least one vascular access device. The vascular access device placement modeling system may communicate an instruction message for placement of the at least one vascular access device based on the at least one first probability to the at least one user device.

In some non-limiting embodiments or aspects, the at least one user device may include the reader device.

In some non-limiting embodiments or aspects, at least one server may include the at least one database and the vascular access device placement modeling system.

In some non-limiting embodiments or aspects, the at least one user device may receive as input at least one of the clinician data, the patient data, the vascular access device data, any combination thereof, and/or the like. Additionally or alternatively, the at least one user device may communicate the input to the at least one database.

According to non-limiting embodiments or aspects, provided is a computer program product for vascular access device placement. In some non-limiting embodiments or aspects, a computer program product for vascular access device placement may include at least one non-transitory computer-readable medium including one or more instructions that, when executed by at least one processor, cause the at least one processor to receive clinician data associated with at least one clinician, and the at least one clinician may include a first clinician. Patient data associated with at least one patient may be received, and the at least one patient may include a first patient. At least one first probability of successful placement of at least one vascular access device by the first clinician at at least one access point of the first patient may be generated based on the patient data associated with the first patient and the clinician data associated with the first clinician. An instruction message for placement of the at least one vascular access device may be communicated based on the at least one first probability to a user device.

Further embodiments or aspects are set forth in the following numbered clauses:

Clause 1: A method for vascular access device placement, comprising: receiving, with at least one processor, clinician data associated with at least one clinician, the at least one clinician comprising a first clinician; receiving, with at least one processor, patient data associated with at least one patient, the at least one patient comprising a first patient; generating, with at least one processor, at least one first probability of successful placement of at least one vascular access device by the first clinician at at least one access point of the first patient based on the patient data associated with the first patient and the clinician data associated with the first clinician; and communicating, with at least one processor, an instruction message for placement of the at least one vascular access device based on the at least one first probability to a device user.

Clause 2: The method of clause 1, wherein the clinician data comprises at least one of years of experience, handedness, level of training, years of degree, whether the clinician is a member of an intravenous (IV) team, salary, a number of attempted placements of vascular access devices, a number of successful placements of the vascular access devices, a number of attempts per successful placement of each vascular access device, a number of attempted placements of the vascular access devices per patient, a vascular access device placement success ratio, an access point for each attempted placement of the vascular access devices, a number of successful placements of the vascular access devices per selectable subperiod over a selectable time period, whether at least one guidance system was used for each attempted placement of the vascular access devices, whether the at least one guidance system was used for each successful placement of the vascular access devices, or any combination thereof.

Clause 3: The method of any preceding clause, wherein the patient data comprises at least one of height, weight, body mass index (BMI), sex, handedness, dominant side, current diagnostic code, one or more comorbidity diagnostic codes, previous intravenous (IV) treatments, skin pallor, skin shade, whether the patient has a tremor or other involuntary movement disorder, whether the patient has a condition requiring restraint or sedation, a categorization as Difficult Intra Venous Access (DIVA) or Adult Difficult Intra Venous Access (A-DIVA), an American Society of Anesthesiologists (ASA) physical status classification, whether the patient has an unplanned indication for surgery, whether the patient has undergone preoperative fasting, palpability of a target vein at the at least one access point, visibility of a target vein at the at least one access point, a known history of difficult peripheral IV cannulation, a dilated vein diameter smaller than a selectable threshold, size of the vascular access device, at least one available access point on at least one extremity of the patient, whether the patient has at least one vascular disease, whether the patient has preoperative hypovolemia, whether the patient has renal insufficiency, a level of fear of needles of the patient, a measure of difficulty of maintaining peripheral IV access once a respective vascular access device is placed, a number of attempted placements of vascular access devices for the patient, a number of successful placements of the vascular access devices for the patient, a number of attempts per successful placement of each vascular access device for the patient, a vascular access device placement success ratio for the patient, an access point for each attempted placement of the vascular access devices for the patient, or any combination thereof.

Clause 4: The method of any preceding clause, wherein generating the at least one first probability comprises generating the at least one first probability using a neural network model, the neural network model comprising at least one neural network trained based on historical clinician data associated with a plurality of clinicians and historical patient data associated with a plurality of patients.

Clause 5: The method of any preceding clause, further comprising, before generating the at least one first probability, training, with at least one processor, the at least one neural network of the neural network model based on the historical clinician data and the historical patient data.

Clause 6: The method of any preceding clause, further comprising: receiving, with at least one processor, first patient access point data associated with the at least one access point of the first patient, wherein generating the at least one first probability comprises generating the at least one first probability based on the patient data associated with the first patient, the clinician data associated with the first clinician, and the first patient access point data.

Clause 7: The method of any preceding clause, wherein the at least one clinician comprises a plurality of clinicians comprising the first clinician, wherein the at least one patient comprises a plurality of patients comprising the first patient, the method further comprising: receiving, with at least one processor, first clinician identification data associated with the first clinician of the plurality of clinicians; and receiving, with at least one processor, first patient identification data associated with the first patient of the plurality of patients.

Clause 8: The method of any preceding clause, further comprising: receiving, with at least one processor, guideline data associated with at least one recommended access point for each of at least one vascular access procedure; receiving, with at least one processor, vascular access device data associated with the at least one vascular access device and cost data associated with supplies required for an attempted placement of the at least one vascular access device; and generating, with at least one processor, the instruction message indicating an instruction to place a first vascular access device of the at least one vascular access devices at a first access point of the at least one access point based on the at least one first probability, the guideline data, the vascular access device data, and the cost data.

Clause 9: The method of any preceding clause, further comprising, before communicating the instruction message: determining, with at least one processor, that the at least one first probability satisfies a threshold; and generating, with at least one processor, the instruction message indicating an instruction for the first clinician to place the at least one vascular access device at a first access point of the at least one access point based on the at least one first probability satisfying the threshold.

Clause 10: The method of any preceding clause, further comprising, before communicating the instruction message: determining, with at least one processor, that the first probability does not satisfy a threshold; and generating, with at least one processor, the instruction message indicating an instruction for at least one intervention for placement of the at least one vascular access device based on the at least one first probability failing to satisfy the threshold, wherein the at least one intervention comprises at least one of a second clinician for placement of the vascular access device, at least one guidance system for placement of the vascular access device, or any combination thereof.

Clause 11: The method of any preceding clause, further comprising, before generating the instruction message: generating at least one of: at least one second probability of successful placement of the at least one vascular access device by the first clinician at the at least one access point of the first patient based on the patient data associated with the first patient, the clinician data associated with the first clinician, and guidance data associated with at least one guidance system; at least one third probability of successful placement of the at least one vascular access device by a second clinician of the at least one clinician at the at least one access point of the first patient based on the patient data associated with the first patient and the clinician data associated with the second clinician; or at least one fourth probability of successful placement of the at least one vascular access device by the second clinician at the at least one access point of the first patient based on the patient data associated with the first patient, the clinician data associated with the second clinician, and the guidance data associated with at least one guidance system.

Clause 12: The method of any preceding clause, further comprising: generating, with at least one processor, at least one first predicted patient satisfaction for an attempted placement of the at least one vascular access device by the first clinician at the at least one access point of the first patient based on the patient data associated with the first patient and the clinician data associated with the first clinician; and generating the instruction message for placement of the at least one vascular access device based on the at least one first probability and the at least one first predicted patient satisfaction, wherein communicating the instruction message comprises communicating the instruction message for placement of the at least one vascular access device based on the at least one first probability and the at least one first predicted patient satisfaction to the user device.

Clause 13: The method of any preceding clause, further comprising: receiving, with at least one processor, input data based on an attempted placement of the at least one vascular access device by the first clinician at the at least one access point of the first patient, the input data comprising at least one of: an update to the patient data associated with the first patient; an update to the clinician data associated with the first clinician; a side of the patient in which the attempted placement of the at least one vascular access device was performed; a first access point of the at least one access point at which the attempted placement of the at least one vascular access device was performed; a visibility of a target vein at the at least one access point; a dilated vein diameter of the target vein at the at least one access point; a palpability of the target vein at the at least one access point; a size of the at least one vascular access device; a type of the at least one vascular access device; a number of attempted placements of the at least one vascular access device; an indication of whether the attempted placement of the at least one vascular access device was successful; a time from when placement of the at least one vascular access device was ordered to the attempted placement of the at least one vascular access device; an indication of whether at least one intervention for placement of the at least one vascular access device was used; a level of fear of the attempted placement of the at least one vascular access device of the patient; a first pain score of the patient during the attempted placement of the at least one vascular access device; a second pain score of the patient after the attempted placement of the at least one vascular access device; a patient satisfaction score for the attempted placement of the at least one vascular access device; or any combination thereof.

Clause 14: The method of any preceding clause, further comprising: generating, with at least one processor, at least one of: at least one predicted cost for placement of the at least one vascular access device; at least one expected time from when placement of the at least one vascular access device was ordered to the successful placement of the at least one vascular access device; at least one first expected pain score of the patient during placement of the at least one vascular access device; at least one second expected pain score of the patient after placement of the at least one vascular access device; at least one difficulty score for placement of the at least one vascular access device at the at least one access point of the first patient; or any combination thereof.

Clause 15: The method of any preceding clause, further comprising: generating, with at least one processor, at least one graphical user interface (GUI) indicating at least one of: an average time from when placement of the at least one vascular access device was ordered to the successful placement of the at least one vascular access device as a function of patient difficulty; a vascular access device placement success ratio as a function of the patient difficulty; a first average pain score during placement of the at least one vascular access device as a function of patient difficulty; a second average pain score after placement of the at least one vascular access device as a function of patient difficulty; an average patient satisfaction score; a summary of placements of vascular access devices; or any combination thereof

Clause 16: A system comprising: at least one clinician identification device, the at least one clinician identification device comprising a first clinician identification device associated with a first clinician; at least one patient identification device, the at least one patient identification device comprising a first patient identification device associated with a first patient; at least one vascular access device, each vascular access device comprising a vascular access device identification device associated therewith; at least one reader device configured to communicate with the at least one clinician identification device, the at least one patient identification device, and each vascular access device identification device, the reader connected to a communication network; at least one user device connected to the communication network; at least one database connected to the communication network, the at least one database configured to: store clinician data associated with a plurality of clinicians, the plurality of clinicians comprising the first clinician; store patient data associated with a plurality of patients, the plurality of patients comprising the first patient, the patient data for the first patient comprising access point data associated with at least one access point of the first patient; and store vascular access device data associated with a plurality of vascular access devices, the plurality of vascular access devices comprising the at least one vascular access device; and a vascular access device placement modeling system connected to the communication network, the vascular access device placement modeling system configured to: receive first clinician identification data associated with the first clinician from the first clinician identification device via the reader device; receive first patient identification data associated with the first patient from the first patient identification device via the reader device; receive vascular access device identification data associated with the at least one vascular access device from the vascular access device identification device associated with each of the at least one vascular access device via the reader device; retrieve the clinician data associated with the first clinician from the at least one database; retrieve the patient data associated with the first patient from the at least one database; retrieve the vascular access device data associated with each of the at least one vascular access device from the at least one database; generate at least one first probability of successful placement of the at least one vascular access device by the first clinician at the at least one access point of the first patient based on the patient data associated with the first patient, the clinician data associated with the first clinician, and the vascular access device data associated with each of the at least one vascular access device; and communicate an instruction message for placement of the at least one vascular access device based on the at least one first probability to the at least one user device.

Clause 17: The system of clause 16, wherein the at least one user device comprises the reader device.

Clause 18: The system of any one of clauses 16 or 17, wherein at least one server comprises the at least one database and the vascular access device placement modeling system.

Clause 19: The system of any one of clauses 16-18, wherein the at least one user device is configured to: receive as input at least one of the clinician data, the patient data, the vascular access device data, or any combination thereof; and communicate the input to the at least one database.

Clause 20: A computer program product for vascular access device placement comprising at least one non-transitory computer-readable medium including one or more instructions that, when executed by at least one processor, cause the at least one processor to: receive clinician data associated with at least one clinician, the at least one clinician comprising a first clinician; receive patient data associated with at least one patient, the at least one patient comprising a first patient; generate at least one first probability of successful placement of at least one vascular access device by the first clinician at at least one access point of the first patient based on the patient data associated with the first patient and the clinician data associated with the first clinician; and communicate an instruction message for placement of the at least one vascular access device based on the at least one first probability to a user device.

Clause 21: A system comprising at least one processor and at least one non-transitory computer-readable medium including one or more instructions that, when executed by the at least one processor, cause the at least one processor to perform the method of any one of clauses 1-15.

Clause 22: At least one non-transitory computer-readable medium including one or more instructions that, when executed by at least one processor, cause the at least one processor to perform the method of any one of clauses 1-15.

These and other features and characteristics of the presently disclosed subject matter, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter. As used in the specification and the claims, the singular form of “a,” “an,” and “the” include plural referents unless the context clearly dictates otherwise.

### BRIEF DESCRIPTION OF THE DRAWINGS

Additional advantages and details of the disclosed subject matter are explained in greater detail below with reference to the exemplary embodiments or aspects that are illustrated in the accompanying figures, in which:

FIG. 1 is a diagram of non-limiting embodiments or aspects of an environment in which systems, methods, and/or products, described herein, may be implemented according to the principles of the presently disclosed subject matter;

FIG. 2 is a diagram of non-limiting embodiments or aspects of components of one or more devices of FIG. 1;

FIG. 3 is a flowchart of non-limiting embodiments or aspects of a process for vascular access device placement according to the principles of the presently disclosed subject matter;

FIG. 4 is a diagram of a non-limiting embodiment or aspect of an implementation of a non-limiting embodiment or aspect of the process shown in FIG. 3 according to the principles of the presently disclosed subject matter;

FIG. 5 is a diagram of a non-limiting embodiment or aspect of an implementation of a non-limiting embodiment or aspect of the process shown in FIG. 3 according to the principles of the presently disclosed subject matter; and

FIGS. 6A-6D are screenshots of exemplary graphical user interfaces of a non-limiting embodiment or aspect of an implementation of a non-limiting embodiment or aspect of the process shown in FIG. 3 according to the principles of the presently disclosed subject matter.

### DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosed subject matter as it is oriented in the drawing figures. However, it is to be understood that the disclosed subject matter may assume various alternative variations and step sequences, except where expressly specified to the contrary. It is also to be understood that the specific devices and processes illustrated in the attached drawings, and described in the following specification, are simply exemplary embodiments or aspects of the disclosed subject matter. Hence, specific dimensions and other physical characteristics related to the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” and/or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “server” may refer to one or more computing devices, such as processors, storage devices, and/or similar computer components that communicate with client devices and/or other computing devices over a network, such as the Internet or private networks, and, in some examples, facilitate communication among other servers and/or client devices. It will be appreciated that various other arrangements are possible. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Non-limiting embodiments or aspects of the disclosed subject matter are directed to systems, methods, and products for vascular access device placement, including, but not limited to, vascular access device placement based on clinician data and patient data. For example, non-limiting embodiments or aspects of the disclosed subject matter provide generating at least one probability of successful placement of at least one vascular access device by a clinician at at least one access point of a patient based on patient data and clinician data and communicating an instruction message for placement of the vascular access device(s) based on the probability (or probabilities) to a user device. Such embodiments or aspects provide techniques and systems that allow for balancing allowing clinicians with less experience and/or training the opportunity to attempt placements of vascular access devices with reducing for unsuccessful attempts for such placements. For example, allowing the initially assigned clinician to attempt to place a vascular access device when the probability of successful placement of the vascular access device satisfies a threshold (e.g., sufficiently high probability) may provide the initially assigned clinician an opportunity for learning (e.g., increasing the initial clinician's experience and/or training), reduce inefficient use of resources (e.g., unnecessarily using an additional clinician and/or a guidance system that may be more appropriately made available to help other patients), and reduce costs (e.g., the price of having additional clinicians and/or additional guidance systems). Additionally or alternatively, instructing the use of at least one intervention (e.g., assigning another physician and/or using a guidance system) for placement of the vascular access device when the probability of successful placement of the vascular access device by the initially assigned clinician does not satisfy the threshold (e.g., probability) may reduce unsuccessful attempts to place vascular access devices, thereby reducing pain for the patient, dissatisfaction of the patient, fear of vascular access devices in the patient, wasted resources (e.g., needing to dispose of the vascular access devices, disinfecting products, bandages, and/or the like), and wasted time. Additionally or alternatively, such embodiments or aspects provide techniques and systems that provide instructions for which access point of multiple access points and/or which vascular access device type of multiple vascular access devices types to use for a particular patient at that particular time (e.g., for a particular procedure ordered and/or the like).

Additionally, non-limiting embodiments or aspects of the disclosed subject matter provide a unified analysis of the clinician's experience (e.g., from clinician data) and a patient's risk factors (e.g., from patient data) to provide the clinician with a customized recommendation (e.g., instruction message) as to whether the patient will be readily cannulated by the clinician and/or whether the patient should be recommended for intervention, e.g., by a more skilled clinician (e.g., another clinician with more experience or specialized role (e.g., IV team)), by using placement aid (e.g., a guidance system such as ultrasound guided placement of the vascular access device), any combination thereof, and/or the like. For example, such a recommendation (e.g., instruction message) may be generated based on the history of attempted and/or successful placements of vascular access devices for the patient, the difficulty of placing such vascular access devices, the expertise of the clinician, the difficulty of maintaining peripheral IV access for the patient once it is obtained, any combination thereof, and/or the like. For example, determining the difficulty of maintaining peripheral IV access for the patient may include analyzing historical data of how long peripheral IV access was maintained for the patient with respect to how long the peripheral IV access was needed, how long the type of vascular access devices (e.g., catheter) is intended to be used (e.g., its life), and/or the like. For the purpose of illustration, a first patient with a peripheral IV access that was dislodged at day four when the peripheral IV access was required for seven days (e.g., thus needing to be re-established) would potentially be higher risk than a second patient who had a peripheral IV access for the full three-day period that such peripheral IV access was required. Moreover, non-limiting embodiments or aspects of the disclosed subject matter allow for the consideration of how difficult each peripheral IV access in the patient's history was (e.g., a difficulty score and/or categorization), a number of attempts at placement of each vascular access devices, the level of experience and/or skill of the clinician (e.g., based on clinician data, such as years of experience, average number of successful placements performed per subperiod (e.g., week) over a time period (e.g., six-month period)), whether a placement aid (e.g., ultrasound guidance, other catheter placement aids, and/or the like) was used, and/or the like.

For the purpose of illustration, in the following description, while the presently disclosed subject matter is described with respect to systems, methods, and products for vascular access device placement, e.g., based on clinician data and/or patient data, one skilled in the art will recognize that the disclosed subject matter is not limited to the illustrative embodiments or aspects. For example, the systems, methods, and products described herein may be used with a wide variety of settings, such as placement of medical devices and/or administration of medical treatments in any setting suitable for using such devices and/or treatments, e.g., external medical devices, implanted medical devices, topical treatments, surgical treatments, any combination thereof, and/or the like.

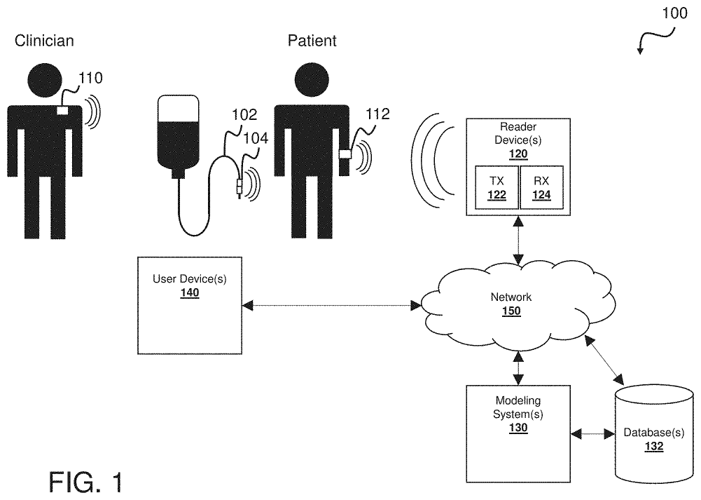

Referring now to FIG. 1, FIG. 1 is a diagram of a non-limiting embodiment or aspect of an environment 100 in which devices, systems, and/or methods, as described herein, may be implemented. As shown in FIG. 1, environment 100 may include medical device 102, medical device identification device 104, clinician identification device 110, patient identification device 112, reader device 120, vascular access device placement modeling system 130, database 132, user device 140, and/or network 150.

Medical device 102 may include at least one device and/or a component thereof configured to be used for medical purposes. For example, medical device 102 may include at least one device (e.g., instrument, apparatus, implement, machine, contrivance, implant, any combination thereof, and/or the like) and/or a component thereof, which may be configured for use in the diagnosis of disease or other conditions and/or in the cure, mitigation, treatment, and/or prevention of disease and/or other conditions (e.g., in a human, other animals, and/or the like). In some non-limiting embodiments or aspects, medical device 102 may include a vascular access device (e.g., intravenous (IV) line, catheter, needle, cannula, and/or the like), a syringe, any combination thereof, and/or the like.

In some non-limiting embodiments or aspects, medical device 102 may include medical device identification device 104. For example, medical device identification device 104 may include at least one machine-readable optical label (e.g., barcode, quick response (QR) code, and/or the like), at least one radio frequency identification (RFID) tag (e.g., an RFID tag with an integrated circuit (IC) storing identification data thereof, a chipless RFID tag, any combination thereof, and/or the like), a computing device (e.g., a portable and/or handheld device (e.g., a personal digital assistant (PDA), a smartphone, a tablet, and/or the like)), and/or the like. In some non-limiting embodiments or aspects, medical device identification device 104 may be affixed to medical device 102 (e.g., printed thereon, attached thereto (e.g., with adhesive and/or the like), any combination thereof, and/or the like). Additionally or alternatively, medical device identification device 104 may be included with (e.g., affixed to, integrated in, part of, any combination thereof, and/or the like) packaging and/or protective covering(s) of medical device 102. In some non-limiting embodiments or aspects, medical device identification device 104 may produce a signal upon opening of the packaging and/or protective covering(s) of medical device 102.

In some non-limiting embodiments or aspects, each type of medical device 102 may have a unique identifier associated therewith. For example, the unique identifier may include a stock keeping unit (SKU), a serial number, a model number, a medical device type identification number, any combination thereof, and/or the like. In some non-limiting embodiments or aspects, the unique identifier for each respective type of medical device 102 may be associated with (e.g., stored on, represented by, mapped to, and/or the like) a respective medical device identification device 104. For example, a first medical device identification device 104 may be associated with a first unique identifier of a first type of medical device 102, a second medical device identification device 104 may be associated with a second unique identifier of a second type of medical device 102, and/or the like.

In some non-limiting embodiments or aspects, medical device 102 may include a mating element. For example, the mating element may include any element configured to be used for mating medical device 102 with a second medical device. In some non-limiting embodiments or aspects, the mating element may include a luer fitting (e.g., a male luer fitting, a female luer fitting, and/or the like).

Clinician identification device 110 may include at least one machine-readable optical label (e.g., barcode, QR code, and/or the like), at least one RFID tag (e.g., an RFID tag with an IC storing identification data thereof, a chipless RFID tag, any combination thereof, and/or the like), a computing device (e.g., a portable and/or handheld device (e.g., a PDA, a smartphone, a tablet, and/or the like)), and/or the like. In some non-limiting embodiments or aspects, clinician identification device 110 may be wearable by a clinician. For example, clinician identification device 110 may include a badge, a bracelet, a watch, glasses, one or more lenses, clothing, and/or the like. Additionally or alternatively, clinician identification device 110 may be included with (e.g., affixed to, integrated in, part of, any combination thereof, and/or the like) user device 140.

In some non-limiting embodiments or aspects, each clinician may have clinician identification data associated therewith. For example, the clinician identification data may include a name, a birthdate, a clinician identifier (e.g., unique identifier, employee number, and/or the like), any combination thereof, and/or the like. For example, the clinician identification data for each respective clinician may be associated with (e.g., stored on, represented by, mapped to, and/or the like) a respective clinician identification device 110. For example, a first clinician identification device 110 may be associated with a first clinician identifier of a first clinician, a second clinician identification device 110 may be associated with a second clinician identifier of a second clinician, and/or the like.

Patient identification device 112 may include at least one machine-readable optical label (e.g., barcode, QR code, and/or the like), at least one RFID tag (e.g., an RFID tag with an IC storing identification data thereof, a chipless RFID tag, any combination thereof, and/or the like), a computing device (e.g., a portable and/or handheld device (e.g., a PDA, a smartphone, a tablet, and/or the like)), and/or the like. In some non-limiting embodiments or aspects, patient identification device 112 may be wearable by a patient. For example, patient identification device 112 may include a badge, a bracelet, a watch, glasses, one or more lenses, clothing, and/or the like.

In some non-limiting embodiments or aspects, each patient may have patient identification data associated therewith. For example, the patient identification data may include a name, a birthdate, a patient identifier (e.g., unique identifier, patient number, account number, and/or the like), any combination thereof, and/or the like. For example, the patient identification data for each respective patient may be associated with (e.g., stored on, represented by, mapped to, and/or the like) a respective patient identification device 112. For example, a first patient identification device 112 may be associated with a first patient identifier of a first patient, a second patient identification device 112 may be associated with a second patient identifier of a second patient, and/or the like.

Reader device 120 may include one or more devices capable of receiving information from and/or communicating information to vascular access device placement modeling system 130, database 132, and/or user device 140 (e.g., via network 150). Additionally or alternatively, each reader device 120 may include a device capable of receiving information from and/or communicating information to other reader devices 120 (e.g., via network 150, another network (e.g., an ad hoc network, a local network, a private network, a virtual private network, and/or the like), and/or any other suitable communication technique). In some non-limiting embodiments or aspects, reader device 120 may be capable of receiving information (e.g., from another reader device 120, medical device identification device 104, clinician identification device 110, patient identification device 112, and/or the like) via a short-range wireless communication connection (e.g., a near-field communication (NFC) communication connection, an RFID communication connection, a Bluetooth® communication connection, a Zigbee® communication connection, and/or the like), and/or communicating information (e.g., to another reader device 120).

In some non-limiting embodiments or aspects, each reader device 120 may include at least one of transmitter 122, receiver 124, and/or any combination thereof. Additionally or alternatively, at least one of transmitter 122, receiver 124, and/or any combination thereof, may be separate from reader device 120, and reader device 120 may include a device capable of receiving information from and/or communicating information to transmitter 122 and/or receiver 124 (e.g., via network 150, another network (e.g., an ad hoc network, a local network, a private network, a virtual private network, and/or the like), and/or any other suitable communication technique).

Transmitter 122 may include at least one transmitter (e.g., at least one device and/or circuit configured to generate and/or transmit electromagnetic waves, such as a signal generator, a radio frequency (RF) transmitter, a microwave transmitter, an analog transmitter, a digital transmitter, any combination thereof, and/or the like). Additionally or alternatively, transmitter 122 may generate alternating current (e.g., RF alternating current, microwave alternating current, and/or the like). In some non-limiting embodiments or aspects, reader device 120 and/or transmitter 122 may include at least one antenna (e.g., antenna element, dipole antenna, and/or the like). Additionally or alternatively, transmitter 122 may apply the alternating current to the antenna, which may be excited by the alternating current to thereby transmit electromagnetic waves (e.g., radio waves, microwaves, and/or the Ike). In some non-limiting embodiments or aspects, transmitter 122 may include one or more devices capable of receiving information from and/or communicating information to reader device 120, vascular access device placement modeling system 130, database 132, and/or user device 140 (e.g., via network 150).

Receiver 124 may include at least one receiver (e.g., at least one device and/or circuit configured to receive electromagnetic waves). In some non-limiting embodiments or aspects, reader device 120 and/or receiver 124 may include at least one antenna (e.g., antenna element, dipole antenna, and/or the like). Additionally or alternatively, the antenna may receive electromagnetic waves to thereby generate alternating currents. Additionally or alternatively, such alternating currents may be applied (e.g., by the antenna) to the receiver, which may extract information therefrom. For example, the receiver may determine which frequency components are present in the received signal (e.g., received electromagnetic waves and/or the like). In some non-limiting embodiments or aspects, receiver 124 may include one or more devices capable of receiving information from and/or communicating information to reader device 120, vascular access device placement modeling system 130, database 132, and/or user device 140 (e.g., via network 150).

In some non-limiting embodiments or aspects, transmitter 122 and/or reader device 120 may transmit an interrogation signal to at least one of medical device identification device 104, clinician identification device 110, patient identification device 112, any combination thereof, and/or the like. Additionally or alternatively, receiver 124 and/or reader device 120 may receive a response signal from at least one of medical device identification device 104, clinician identification device 110, patient identification device 112, any combination thereof, and/or the like. The response signal may be associated with the identification data (e.g., unique identifier associated with a respective type of medical device 102, clinician identification data associated with a respective clinician, patient data associated with a respective patient, and/or the like) of the medical device identification device 104, clinician identification device 110, patient identification device 112, and/or the like, respectively. In some non-limiting embodiments or aspects, receiver 124 and/or reader device 120 may communicate response signal data associated with the response signal (e.g., to vascular access device placement modeling system 130, database 132, and/or user device 140 via network 150). For example, receiver 124 and/or reader device 120 may include a communication interface for such communication.

In some non-limiting embodiments or aspects, a plurality of reader devices 120 may be included. Additionally or alternatively, each reader device 120 may be disposed at a location (e.g., known location, predetermined location, selectable location, preselected location, any combination thereof, and/or the like) within at least one facility (e.g., at least one building, at least one ward, at least one hospital, and/or the like). For example, the location of each reader device 120 may be different than the location of all other reader devices 120 (e.g., each reader device 120 may be at a different location within the site(s)). In some non-limiting embodiments or aspects, a location of medical device 102, clinician identification device 110, and/or patient identification device 112 may be determined (e.g., by reader device 120, vascular access device placement modeling system 130, database 132, and/or user device 140, and/or the like) based on which reader device 120 of the plurality of reader devices 120 detects medical device identification device 104, clinician identification device 110, and/or patient identification device 112, respectively.

In some non-limiting embodiments or aspects, transmitter 122 and/or receiver 124 may be included in a single device (e.g., reader device 120 and/or the like). Additionally or alternatively, transmitter 122 and/or receiver 124 may share a single antenna (e.g., antenna of reader device 120 and/or the like). In some non-limiting embodiments or aspects, transmitter 122 and/or receiver 124 may be separate devices.

In some non-limiting embodiments or aspects, reader device 120 may be included with (e.g., affixed to, integrated in, part of, any combination thereof, and/or the like) user device 140.

Vascular access device placement modeling system 130 may include one or more devices capable of receiving information from and/or communicating information to reader device 120, database 132, and/or user device 140 (e.g. via network 150). For example, vascular access device placement modeling system 130 may include one or more computing devices such as a server, a group of servers, a portable and/or handheld device (e.g., a computer, a laptop, a PDA, a smartphone, a tablet, and/or the like), a desktop computer, and/or the like. In some non-limiting embodiments or aspects, vascular access device placement modeling system 130 may be associated with a facility, as described herein. In some non-limiting embodiments or aspects, vascular access device placement modeling system 130 may be in communication with at least one data storage device (e.g., database 132 and/or the like), which may be local or remote to vascular access device placement modeling system 130. In some non-limiting embodiments or aspects, vascular access device placement modeling system 130 may be capable of receiving information from, storing information in, communicating information to, or searching information stored in the data storage device(s) (e.g., database(s) 132 and/or the like).

In some non-limiting embodiments or aspects, vascular access device placement modeling system 130 may be connected to network 150. Additionally or alternatively, vascular access device placement modeling system 130 may receive first clinician identification data associated with a clinician from clinician identification device 110 via reader device 120, receive patient identification data associated with a patient from patient identification device 112 via reader device 120, receive medical device identification data (e.g., vascular access device identification data and/or the like) associated with medical device 102 (e.g., a vascular access device and/or the like) from vascular access device identification device 104 via reader device 120, any combination thereof, and/or the like, as described herein. In some non-limiting embodiments or aspects, vascular access device placement modeling system 130 may retrieve clinician data associated with the clinician from database(s) 132, retrieve patient data associated with the patient from database(s) 132, retrieve medical device data (e.g., vascular access device data) associated with medical device 102 (e.g., the vascular access device) from the database(s) 132, any combination thereof, and/or the like, as described herein. In some non-limiting embodiments or aspects, vascular access device placement modeling system 130 may generate at least one first probability of successful use of medical device 102 (e.g., placement of the vascular access device) by the clinician at at least one access point of the patient based on the patient data, the clinician data, and the medical device data (e.g., vascular access device data), any combination thereof, and/or the like, as described herein. In some non-limiting embodiments or aspects, vascular access device placement modeling system 130 may communicate an instruction message for use of medical device 102 (e.g., placement of the vascular access device) based on the first probability (or probabilities) to user device 140, as described herein.

Database 132 may include one or more devices capable of receiving information from and/or communicating information to reader device 120, vascular access device placement modeling system 130, and/or user device 140 (e.g. via network 150). Additionally or alternatively, database 132 may include a data storage device, which may be local or remote to vascular access device placement modeling system 130. In some non-limiting embodiments or aspects, database 132 may be integrated with (e.g., completely, partially, and/or the like) and/or directly connected to intelligent industrial assistant 102. Additionally or alternatively, database 132 may be implemented (e.g., completely, partially, and/or the like) separate from vascular access device placement modeling system 130. For example, database 132 may be a separate device and/or system from vascular access device placement modeling system 130. In some non-limiting embodiments or aspects, database 132 and vascular access device placement modeling system 130 may be associated with the same facility, as described herein. In some non-limiting embodiments or aspects, database 132 may include a computing device, such as a server, a group of servers, and/or other like devices. Additionally or alternatively, database 132 may include at least one structured query language (SQL) database, at least one non-SQL database, any combination thereof, and/or the like. In some non-limiting embodiments or aspects, database 132 may be capable of retrieving information from, storing information in, communicating information to, or searching information stored in the data storage device.

In some non-limiting embodiments or aspects, database 132 may be connected to network 150. In some non-limiting embodiments or aspects, database 132 may store clinician data associated with a plurality of clinicians, patient data associated with a plurality of patients, access point data associated with at least one access point (e.g., for each patient), medical device data (e.g., vascular access device data) associated with a plurality of medical devices 102 (e.g., vascular access devices), clinician identification data associated with each clinician of the plurality of clinicians, patient identification data associated with each patient of the plurality of patients, medical device identification data (e.g., vascular access device identification data) associated with each of the plurality of medical devices 102 (e.g., vascular access devices), guideline data associated with access point(s) for each of at least one vascular access procedure, cost data associated with supplies (e.g., additional supplies other than the medical device 102) required for an attempted use of each medical device 102 (e.g., attempted placement of each vascular access device), guidance data associated with at least one guidance system, scheduling data associated with a schedule for each clinician of the plurality of clinicians, preferences data associated with selectable preferences for at least one facility, any combination thereof, and/or the like, as described herein.

In some non-limiting embodiments or aspects, database 132 may include a clinician database (e.g., for storing clinician data), a patient database (e.g., for storing patient data), a salary database, a scheduling database, an inventory database, an electronic medical record (EMR) and/or electronic health record (ERR) database, a hospital preferences database, a guidelines database, any combination thereof, and/or the like.

User device 140 may include one or more devices capable of receiving information from and/or communicating information to reader device 120, vascular access device placement modeling system 130, and/or database 132 (e.g., via network 150). For example, user device 140 may include a computing device (e.g., a portable and/or handheld device (e.g., a computer, a laptop, a PDA, a smartphone, a tablet, and/or the like), a desktop computer, a server, a group of servers, and/or other like devices). In some non-limiting embodiments or aspects, user device 140 may include at least one input component (e.g., a component that permits user device 140 to receive information (e.g., via user input), such as a touch screen display, a keyboard, a keypad, a mouse, a button, a switch, a microphone, a camera, and/or the like). Additionally or alternatively, user device 140 may include at least one output component (e.g., a component that provides output information from user device 140, such as a display, a speaker, one or more light-emitting diodes (LEDs), an antenna, and/or the like).

Network 150 may include at least one communication network. For example, network 150 may include a wired network, a wireless network, a cellular network (e.g., a long-term evolution (LTE) network, a third generation (3G) network, a fourth generation (4G) network, a fifth generation (5G) network, a code division multiple access (CDMA) network, and/or the like), a public land mobile network (PLMN), a local area network (LAN), a wide area network (WAN), a metropolitan area network (MAN), a telephone network (e.g., the public switched telephone network (PSTN)), a private network (e.g., a private network associated with a facility), an ad hoc network, an intranet, the Internet, a fiber optic-based network, a computer network, a cloud computing network, and/or the like, and/or a combination of these or other types of networks.

The number and arrangement of systems, devices, and/or networks shown in FIG. 1 are provided as an example. There may be additional systems, devices, and/or networks; fewer systems, devices, and/or networks; different systems, devices, and/or networks; and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices. Additionally or alternatively, a set of systems (e.g., one or more systems) or a set of devices (e.g., one or more devices) of environment 100 may perform one or more functions described as being performed by another set of systems or another set of devices of environment 100.

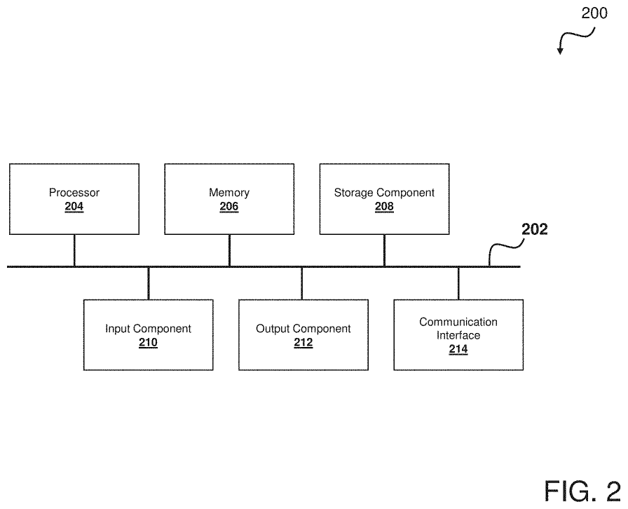

Referring now to FIG. 2, FIG. 2 is a diagram of example components of a device 200. Device 200 may correspond to medical device 102, medical device identification device 104, clinician identification device 110, patient identification device 112, reader device 120, one or more devices of vascular access device placement modeling system 130, one or more devices of database 132, and/or user device 140. In some non-limiting embodiments or aspects, medical device 102, medical device identification device 104, clinician identification device 110, patient identification device 112, reader device 120, vascular access device placement modeling system 130, database 132, and/or user device 140 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, firmware, and/or any combination thereof. For example, processor 204 may include a processor (e.g., a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like), and/or the like, which can be programmed to perform a function. Memory 206 may include random access memory (RAM), read-only memory (ROM), and/or another type of dynamic or static storage device (e.g., flash memory, magnetic memory, optical memory, and/or the like) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, a camera, and/or the like). Additionally or alternatively, input component 210 may include an antenna for receiving electromagnetic radiation, a sensor for sensing information (e.g., a global positioning system (GPS) component, an accelerometer, a gyroscope, an actuator, and/or the like), and/or the like. Output component 212 may include a component that provides output information from device 200 (e.g., an antenna for transmitting electromagnetic radiation, a display, a speaker, one or more light-emitting diodes (LEDs), and/or the like).

Communication interface 214 may include a transceiver-like component (e.g., a transceiver, a receiver and transmitter that are separate, and/or the like) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a Bluetooth® interface, a Zigbee® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

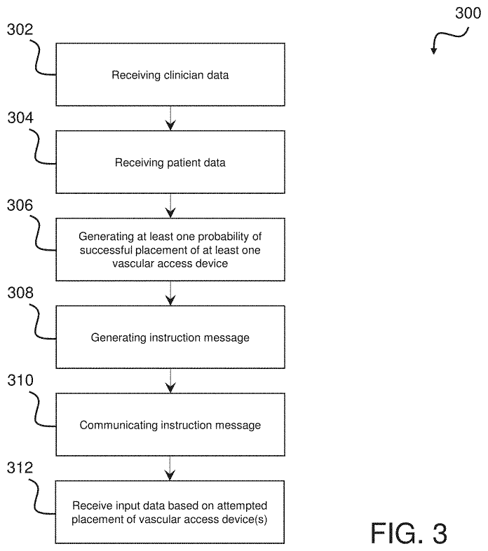

Referring now to FIG. 3, FIG. 3 is a flowchart of a non-limiting embodiment of a process 300 for vascular access device placement. In some non-limiting embodiments or aspects, one or more of the steps of process 300 may be performed (e.g., completely, partially, and/or the like) by vascular access device placement modeling system 130 (e.g., one or more devices of vascular access device placement modeling system 130). In some non-limiting embodiments or aspects, one or more of the steps of process 300 may be performed (e.g., completely, partially, and/or the like) by another system, another device, another group of systems, or another group of devices, separate from or including vascular access device placement modeling system 130, such as medical device 102, medical device identification device 104, clinician identification device 110, patient identification device 112, reader device 120, database 132, user device 140, and/or the like. In some non-limiting embodiments, a vascular access device may be the same as or similar to medical device 102.

As shown in FIG. 3, at step 302, process 300 may include receiving clinician data. For example, vascular access device placement modeling system 130 may receive clinician data associated with at least one clinician (e.g., a plurality of clinicians and/or the like). In some non-limiting embodiments or aspects, the at least one clinician may include a first clinician (e.g., assigned to care for a first patient).

In some non-limiting embodiments or aspects, the clinician data may include background information for the clinician, such as years of experience, handedness, level of training, years of degree, whether the clinician is a member of an intravenous (IV) team, salary, any combination thereof, and/or the like. Additionally or alternatively, the clinician data may include historical vascular access device placement data for the clinician, such as a number of attempted placements of vascular access devices, a number of successful placements of the vascular access devices, a number of attempts per successful placement of each vascular access device, a number of attempted placements of the vascular access devices per patient, a vascular access device placement success ratio, an access point for each attempted placement of the vascular access devices, a number of successful placements of the vascular access devices per selectable subperiod (e.g., per day, per week, and/or the like) over a selectable time period (e.g., a month, a six-month period, one year, and/or the like), whether at least one guidance system was used for each attempted placement of the vascular access devices, whether the at least one guidance system was used for each successful placement of the vascular access devices, any combination thereof, and/or the like.

In some non-limiting embodiments or aspects, vascular access device placement modeling system 130 may receive (e.g., retrieve) clinician data from database 132. Additionally or alternatively, at least some of the clinician data may be inputted via user device 140, and such data may be communicated to vascular access device placement modeling system 130 and/or database 132.

In some non-limiting embodiments or aspects, the at least one clinician may include a plurality of clinicians. Additionally or alternatively, vascular access device placement modeling system 130 may receive first clinician identification data associated with the first clinician from a first clinician identification device 110 via reader device 120, as described herein. In some non-limiting embodiments or aspects, vascular access device placement modeling system 130 may receive (e.g., retrieve) the clinician data associated with the first clinician (e.g., from database 132) based on the first clinician identification data.

As shown in FIG. 3, at step 304, process 300 may include receiving patient data. For example, vascular access device placement modeling system 130 may receive patient data associated with at least one patient (e.g., a plurality of patients and/or the like). In some non-limiting embodiments or aspects, the at least one patient may include a first patient (e.g., a first patient to whom a first clinician was assigned).

In some non-limiting embodiments or aspects, the patient data may include physiological data for the patient, such as height, weight, body mass index (BMI), sex, handedness, dominant side, current diagnostic code, one or more comorbidity diagnostic codes (e.g., Diabetes mellitus, cancer, IV drug abuse, alcohol abuse, vascular disease, hypovolemia, smoking, renal insufficiency, any combination thereof, and/or the like), skin pallor, skin shade, whether the patient has a tremor or other involuntary movement disorder (e.g., Parkinson's disease and/or the like), whether the patient has a condition requiring restraint or sedation, an American Society of Anesthesiologists (ASA) physical status classification, whether the patient has an unplanned indication for surgery, whether the patient has undergone preoperative fasting, whether the patient has renal insufficiency, any combination thereof, and/or the like. Additionally or alternatively, the patient data may include historical vascular access device placement data for the patient, such as previous intravenous (IV) treatments (e.g., prior IV chemo administration, other IV administration of a vesicant drug, any combination thereof, and/or the like), a categorization as Difficult Intra Venous Access (DIVA) or Adult Difficult Intra Venous Access (A-DIVA), a known history of difficult peripheral IV cannulation, a measure of difficulty of maintaining peripheral IV access once a respective vascular access device is placed (e.g., based on analyzing historical data of how long peripheral IV access was maintained for the patient with respect to how long the peripheral IV access was needed, how long the type of vascular access devices (e.g., catheter) is intended to be used (e.g., its life), and/or the like), a number of attempted placements of vascular access devices for the patient, a number of successful placements of the vascular access devices for the patient, a number of attempts per successful placement of each vascular access device for the patient, a vascular access device placement success ratio for the patient, an access point for each attempted placement of the vascular access devices for the patient, any combination thereof, and/or the like. For the purpose of illustration, a first patient with a peripheral IV access that was dislodged at day four when the peripheral IV access was required for seven days (e.g., thus needing to be re-established) would potentially be higher risk than a second patient who had a peripheral IV access for the full three-day period that such peripheral IV access was required. Additionally or alternatively, the patient data may include access point data associated with at least one access point of the patient, such as palpability of a target vein at each access point, visibility of a target vein at each access point, a dilated vein diameter smaller than a selectable threshold (e.g., dilated vein diameter less than 2 mm and/or the like), at least one available access point on at least one extremity of the patient, whether the patient has at least one vascular disease, whether the patient has preoperative hypovolemia, any combination thereof, and/or the like. Additionally or alternatively, the patient data may include a level of fear of needles of the patient, a level of satisfaction of the patient for at least one previous attempted placement of at least one vascular access device, a size of the vascular access device(s) to be used for the patient, any combination thereof, and/or the like.

In some non-limiting embodiments or aspects, the at least one patient may include a plurality of patients. Additionally or alternatively, vascular access device placement modeling system 130 may receive first patient identification data associated with the first patient from a first patient identification device 112 via reader device 120, as described herein. In some non-limiting embodiments or aspects, vascular access device placement modeling system 130 may receive (e.g., retrieve) the patient data associated with the first patient (e.g., from database 132) based on the first patient identification data.

As shown in FIG. 3, at step 306, process 300 may include generating at least one probability, e.g., of successful placement of at least one vascular access device. For example, vascular access device placement modeling system 130 may generating at least one first probability of successful placement of at least one vascular access device by the first clinician at at least one access point of the first patient based on the patient data associated with the first patient, the clinician data associated with the first clinician, any combination thereof, and/or the like. For example, vascular access device placement modeling system 130 may generate a respective first probability of successful placement of each of a plurality of vascular access devices by the first clinician at each of a plurality of access points of the first patient based on the patient data associated with the first patient, the clinician data associated with the first clinician, any combination thereof, and/or the like. In some non-limiting embodiments, each first probability of successful placement of the vascular access device may include a probability of successful placement of the respective vascular access device on a first attempt to place the vascular access device (e.g., first stick).

In some non-limiting embodiments or aspects, generating the at least one first probability may include generating the at least one first probability using at least one predictive model (e.g., a neural network model, a machine learning model, data mining model, a statistical model, a regression model (e.g., for an A-DIVA patient, linear regression analysis), any combination thereof, and/or the like). In some non-limiting embodiments or aspects, generating the at least one first probability may include generating the at least one first probability using a neural network model. For example, the neural network model may include at least one neural network trained based on historical clinician data associated with a plurality of clinicians and historical patient data associated with a plurality of patients. Additionally or alternatively, at least some of the historical clinician data and the historical patient data may be associated with known outcomes (e.g., attempted placements of at least one vascular access device, successful placements of at least one vascular access device, failed placements of at least on vascular access device, any combination thereof, and/or the like). In some non-limiting embodiments or aspects, before generating the at least one first probability, vascular access device placement modeling system 130 may train the at least one neural network of the neural network model based on the historical clinician data and the historical patient data (e.g., and the known outcomes associated therewith).

In some non-limiting embodiments or aspects, the predictive model (e.g., neural network model, machine learning model, data mining model, statistical model, regression model, and/or the like) may automatically use the most recent data (e.g., most recent/up-to-date clinician data, patient data, and/or the like, as stored by vascular access device placement modeling system 130 and/or database 132) for generating the probabilities (e.g., at least one first probability and/or the like) for each placement of at least one vascular access device. As such, the most up-to-date and best (e.g., most recent, highest reliability, and/or the like) predictive value information (e.g., data) may be used to generate the probabilities and/or generate instruction messages based thereon for the clinician. Additionally, the accuracy of the generated probabilities may improve over time, e.g., as more data (e.g., clinician data, patient data, and/or the like) becomes available.

In some non-limiting embodiments or aspects, as a clinician gains more experience with less challenging (e.g., low difficulty score) placements of vascular access devices (e.g., cannulations) and/or demonstrates success with such placements, the probability of successful placement of vascular access devices that are more challenging (e.g., higher difficulty score) by the clinician may increase. Additionally or alternatively, vascular access device placement modeling system 130 may instruct the clinician to attempt such more challenging placements of vascular access devices. Thus, the clinician's level of experience and/or training may increase. Additionally or alternatively, if a clinician fails to successfully place a vascular access device, the probability of successful placement of vascular access devices by the clinician may decrease, and vascular access device placement modeling system 130 may instruct the clinician not to attempt certain (e.g., more challenging) placements of vascular access devices and/or to use at least one intervention (e.g., a second clinician, a guidance system, any combination thereof, and/or the like). In some non-limiting embodiments or aspects, probability of successful placement of vascular access devices by the clinician may therefore constantly update in real time based on the most recent data (e.g., clinician data, patient data, and/or the like) available.

In some non-limiting embodiments or aspects, vascular access device placement modeling system 130 may receiving first patient access point data associated with the at least one access point of the first patient (e.g., as part of the patient data, separately from the patient data, and/or the like). For example, access point data may include palpability of a target vein at the at least one access point, visibility of a target vein at the at least one access point, a known history of difficult peripheral IV cannulation, a dilated vein diameter smaller than a selectable threshold, at least one available access point on at least one extremity of the patient, and/or the like. Additionally or alternatively, access point data may include suggested or prescribed access points from industry standards, guidelines, and/or the like. In some non-limiting embodiments or aspects, generating the at least one first probability may include generating the at least one first probability based on the patient data associated with the first patient, the clinician data associated with the first clinician, and the first patient access point data.

In some non-limiting embodiments or aspects, vascular access device placement modeling system 130 may generate at least one additional probability of successful placement of at least one vascular access device using the at least one intervention. For example, vascular access device placement modeling system 130 may generate at least one second probability of successful placement of the at least one vascular access device by the first clinician at the first access point of the first patient based on the patient data associated with the first patient, the clinician data associated with the first clinician, guidance data associated with at least one guidance system, any combination thereof, and/or the like. Additionally or alternatively, vascular access device placement modeling system 130 may generate at least one third probability of successful placement of the at least one vascular access device by a second clinician at the first access point of the first patient based on the patient data associated with the first patient, the clinician data associated with the second clinician, any combination thereof, and/or the like. Additionally or alternatively, vascular access device placement modeling system 130 may generate at least one fourth probability of successful placement of the at least one vascular access device by the second clinician at the first access point of the first patient based on the patient data associated with the first patient, the clinician data associated with the second clinician, the guidance data associated with at least one guidance system, any combination thereof, and/or the like. In some non-limiting embodiments or aspects, each probability (e.g., second probability, third probability, fourth probability, and/or the like) of successful placement of the vascular access device may include a probability of successful placement of the vascular access device on a first attempt to place the vascular access device (e.g., first stick). In some non-limiting embodiments or aspects, the second clinician may include a specialist, such as a member of an IV team. In some non-limiting embodiments or aspects, the guidance system may include an ultrasound system, at least one other catheter placement aid, any combination thereof, and/or the like.

As shown in FIG. 3, at step 308, process 300 may include generating an instruction message. For example, vascular access device placement modeling system 130 may generate an instruction message for placement of the at least one vascular access device based on the at least one first probability.

In some non-limiting embodiments or aspects, vascular access device placement modeling system 130 may determine that the at least one first probability satisfies a threshold. Additionally or alternatively, vascular access device placement modeling system 130 may generate an instruction message indicating an instruction for the first clinician to place the at least one vascular access device at a first access point of the at least one access point based on the at least one first probability satisfying the threshold.

In some non-limiting embodiments or aspects, vascular access device placement modeling system 130 may determine that the first probability does not satisfy a threshold. Additionally or alternatively, vascular access device placement modeling system 130 may generate an instruction message indicating an instruction for at least one intervention for placement of the at least one vascular access device based on the at least one first probability failing to satisfy the threshold. For example, the intervention(s) may include at least one of a second clinician for placement of the vascular access device, at least one guidance system for placement of the vascular access device, or any combination thereof, and/or the like, as described herein. In some non-limiting embodiments, vascular access device placement modeling system 130 may determine which intervention(s) to instruct based on the at least on additional probability (e.g., second probability, third probability, fourth probability, and/or the like), as described herein.

In some non-limiting embodiments or aspects, the thresholds described herein may be selectable. For example, each threshold may be set for each facility (e.g., ward, hospital, and/or the like) based on preferences data, industry standards, guidelines, any combination thereof, and/or the like. In some non-limiting embodiments, a user (e.g., facility administrator, a clinician, and/or the like) may input preferences (e.g., for the thresholds described herein) via user device 140, and preferences data associated with such preferences may be communicated, e.g., to vascular access device placement modeling system 130 and/or database 132. In some non-limiting embodiments or aspects, the preferences data may further include optimization preferences, e.g., selection and/or adjustment of the thresholds based on balancing training less experienced clinicians (e.g., new staff and/or the like), decreasing costs of placements of vascular access devices, improving patient satisfaction, a level of fear of needles of the patient, reducing patient pain, conserving resources (e.g., additional clinicians and/or guidance systems), reducing time (e.g., from the patient entering the facility and/or from the ordering of a vascular access device/procedure to the placement of such vascular access device), emergent status of the patient, any combination thereof, and/or the like. For example, the optimization preferences may include weighting data and/or other formulas for the aforementioned items and/or any combination thereof.

In some non-limiting embodiments or aspects, for the purpose of illustration, the preferences data (e.g., optimization preferences and/or the like) may include and/or take into account a level of fear of needles of the patient, and the threshold for instructing at least one intervention (e.g., a second clinician that is more experienced, a specialist, and/or the like) may be selected to improve the probability of successful placement of the vascular access device (e.g., improve first stick success) for patients for whom their fear of needles may significantly impact their care experience and/or satisfaction (e.g., patient satisfaction score and/or the like). Additionally or alternatively, the degree (e.g., weighting and/or the like) to which patient's fear and potential impact on satisfaction are taken into account may be selected for each facility, as described herein. Additionally or alternatively, the degree (e.g., weighting and/or the like) to which patient's fear and potential impact on satisfaction are taken into account may be selected based on retrospective analysis, e.g., to understand how large an impact first stick success and/or number of stick attempts has on different patients who self-identify as having different levels of fear of needles, and the threshold may be adjusted based on the retrospective analysis. Additionally or alternatively, patient data may include documentation of negative feedback based on previous failed stick attempts and/or patient feedback on prior visits to adjust the threshold for when at least one intervention is instructed.

In some non-limiting embodiments or aspects, vascular access device placement modeling system 130 may receive guideline data associated with at least one recommended access point for each of at least one vascular access procedure. Additionally or alternatively, vascular access device placement modeling system 130 may receive vascular access device data associated with the at least one vascular access device, cost data associated with supplies required for an attempted placement of the at least one vascular access device, any combination thereof, and/or the like. For example, cost data may include a price of each respective vascular access devices (e.g., catheter, needle, and/or the like), a price for supplies used with the vascular access device (e.g., alcohol swabs, chlorhexidine gluconate (CHG) wipes, other disinfecting products, bandages, any combination thereof, and/or the like), any combination thereof, and/or the like. In some non-limiting embodiments or aspects, vascular access device placement modeling system 130 may generate an instruction message indicating an instruction to place a first vascular access device at a first access point based on the at least one first probability, the at least one additional probability (e.g., second probability, third probability, fourth probability, and/or the like), the guideline data, the vascular access device data, the cost data, any combination thereof, and/or the like. For example, vascular access device placement modeling system 130 may determine which vascular access device, which access point, which clinician, which intervention(s), which combination thereof, and/or the like provides an increased (e.g., highest) probability of successful placement. For the purpose of illustration, vascular access device placement modeling system 130 may determine which clinician should attempt to place a vascular access device at a particular access point based on a clinician having an improved probability of successful placement on a respective side of the patient (e.g., left or right side) based on the handedness of the clinician (e.g., left or right handed). In some non-limiting embodiments or aspects, vascular access device placement modeling system 130 may generate the instruction message further based on timing, how long the type of vascular access devices (e.g., catheter) is intended to be used (e.g., its life, longevity, and/or the like), preferences data, any combination thereof, and/or the like.

In some non-limiting embodiments or aspects, vascular access device placement modeling system 130 may generate at least one first predicted patient satisfaction for an attempted placement of the at least one vascular access device by the first clinician at the at least one access point of the first patient based on the patient data associated with the first patient and the clinician data associated with the first clinician. Additionally or alternatively, vascular access device placement modeling system 130 may generate the instruction message for placement of the at least one vascular access device based on the at least one first probability and the at least one first predicted patient satisfaction.

In some non-limiting embodiments or aspects, vascular access device placement modeling system 130 may generate at least one of the following: at least one predicted cost for placement of the at least one vascular access device (e.g., expected total cost of administering sticks under different conditions (e.g., access point(s), clinician(s), catheter type(s), intervention(s), guidance system(s), and/or the like)), at least one expected time from when placement of the at least one vascular access device was ordered to the successful placement of the at least one vascular access device (e.g., expected time to achieve catheter access based on each clinician (e.g., first clinician, second clinician, and/or the like) and/or the like), at least one first expected pain score of the patient during placement of the at least one vascular access device (e.g., based on each clinician (e.g., first clinician, second clinician, and/or the like) and/or the like), at least one second expected pain score of the patient after placement of the at least one vascular access device (e.g., based on each clinician (e.g., first clinician, second clinician, and/or the like) and/or the like), at least one difficulty score for placement of the at least one vascular access device at the at least one access point of the first patient (e.g., score for difficulty of stick performed and/or the like), any combination thereof, and/or the like.

In some non-limiting embodiments or aspects, the disclosed subject matter (e.g., vascular access device placement modeling system 130 and/or the like) may improve patient outcomes (e.g., reducing pain, increasing satisfaction, reducing time, and/or the like), reduce costs (e.g., decrease utilization of vascular access devices and/or supplies associated therewith, as less of such items will be wasted since the instructions tell the clinician how to proceed with improved probabilities of successful placement). Additionally or alternatively, the disclosed subject matter may enable administrators and/or clinicians to evaluate the relative costs of having a more experienced clinician and/or specialist (e.g., dedicated IV team member) intervene and/or of using other interventions (e.g., guidance systems), e.g., compared with the costs of having the initial clinician attempt placement of the vascular access device (e.g., use of materials that may be wasted if the clinician fails to have first stick success). In some non-limiting embodiments or aspects, vascular access device placement modeling system 130 may be set to auto-optimize costs based on such evaluations (e.g., using salary data for clinicians, cost data for vascular access devices and/or guidance systems, and/or the like) and/or based on other clinician data (e.g., level of experience, education level, etc.), preferences data (e.g., thresholds and/or the like), any combination thereof, and/or the like. Additionally or alternatively, each facility may set preferences for balancing the desire to increase the probability of successful placement (e.g., first stick success) in the near term with the desire for increasing the level of experience for clinicians and/or cost savings from having more experienced clinicians in the longer term.

As shown in FIG. 3, at step 310, process 300 may include communicating the instruction message. For example, vascular access device placement modeling system 130 may communicate the instruction message for placement of the at least one vascular access device based on the at least one first probability to user device 140.

In some non-limiting embodiments or aspects, user device 140 may generate (e.g., render, display, and/or the like) at least one graphical user interface (GUI). In some non-limiting embodiments or aspects, the GUI may indicate at least one metric (e.g., performance metric) selected from the following: an average time from when placement of the at least one vascular access device was ordered to the successful placement of the at least one vascular access device as a function of patient difficulty (e.g., time (e.g., in minutes) for catheter placement by a clinician as a function of patient difficulty), a vascular access device placement success ratio as a function of the patient difficulty (e.g., first stick success over time and based on patient difficulty]), a first average pain score during placement of the at least one vascular access device as a function of patient difficulty, a second average pain score after placement of the at least one vascular access device as a function of patient difficulty, an average patient satisfaction score, a summary of placements of vascular access devices (e.g., a detailed history of IV lines placed for a patient), any combination thereof, and/or the like. Additionally or alternatively, any of (e.g., each of) such metrics may be displayed as a function of time (e.g., for each selectable subperiod (e.g., each day, each week, each month, and/or the like) over a selectable time period (e.g., a number of days, a number of weeks, a number of months, and/or the like)). In some non-limiting embodiments or aspects, these metrics (and/or a subset thereof) may be displayed for a selected clinician. Additionally or alternatively, these metrics (and/or a subset thereof) may be displayed for a selected facility (e.g., ward, hospital, and/or the like). Additionally or alternatively, these metrics (and/or a subset thereof) may be displayed with corresponding metrics from at least one other facility (e.g., a peer hospital, all hospitals, a subset of hospitals (e.g., a quartile of peer hospitals, a median of peer hospitals, a top or bottom half of peer hospitals, a quartile of all hospitals, a median of all hospitals, a top or bottom half of all hospitals, and/or the like), any combination thereof, and/or the like).

In some non-limiting embodiments, the GUI may be web-based (e.g., viewable with a web browser on user device 140). Additionally or alternatively, the GUI may be app-based (e.g., viewable using an application on user device 140, such as a personal computer (PC) application, a tablet application, a mobile phone application, any combination thereof, and/or the like).

As shown in FIG. 3, at step 312, process 300 may include receiving input data, e.g., based on attempted placement of the vascular access device(s). For example, vascular access device placement modeling system 130 may receive input data (e.g., vascular access attempt data) based on an attempted placement of the at least one vascular access device by the first clinician at the at least one access point of the first patient. In some non-limiting embodiments or aspects, the input data (e.g., vascular access attempt data) may be inputted via user device 140 (e.g., by the clinician and/or the like). Additionally or alternatively, at least some of the input data (e.g., vascular access attempt data) may be received automatically, e.g., via reader device 120. For example, reader device 120 may detect at least one of clinician identification device 110 (e.g., based on receiving clinician identification data from clinician identification device 110, as described herein), patient identification device 112 (e.g., based on receiving patient identification data from patient identification device 112, as described herein), vascular access device identification device 104 (e.g., based on receiving vascular access device identification data from vascular access device identification device 104, as described herein), any combination thereof, and/or the like. Additionally or alternatively, reader device 120 may communicate such data to vascular access device placement modeling system 130 and/or database 132, as described herein. In some non-limiting embodiments or aspects, medical device identification device 104 may produce a signal (e.g., that may be detected by reader device 120 and/or communicated from reader device 120 to vascular access device placement modeling system 130 and/or database 132) upon opening of the packaging and/or protective covering(s) of a vascular access device, and input data (e.g., vascular access attempt data) may be recorded (e.g., by vascular access device placement modeling system 130 and/or database 132 based on receiving the signal. Additionally or alternatively, reader device 120 may receive and/or communicate to vascular access device placement modeling system 130 and/or database 132 (e.g., with the signal) clinician identification data from clinician identification device 110 and/or patient identification data from patient identification device 112.

In some non-limiting embodiments or aspects, the input data (e.g., vascular access attempt data) may include at least one of the following: an update to the patient data associated with the first patient, an update to the clinician data associated with the first clinician, a side of the patient in which the attempted placement of the at least one vascular access device was performed, a first access point of the at least one access point at which the attempted placement of the at least one vascular access device was performed, a visibility of a target vein at the at least one access point, a dilated vein diameter of the target vein at the at least one access point, a palpability of the target vein at the at least one access point, a size of the at least one vascular access device, a type of the at least one vascular access device, a number of attempted placements of the at least one vascular access device, an indication of whether the attempted placement of the at least one vascular access device was successful, a time from when placement of the at least one vascular access device was ordered to the attempted placement of the at least one vascular access device, an indication of whether at least one intervention for placement of the at least one vascular access device was used, a level of fear of the attempted placement of the at least one vascular access device of the patient, a first pain score of the patient during the attempted placement of the at least one vascular access device, a second pain score of the patient after the attempted placement of the at least one vascular access device, a patient satisfaction score for the attempted placement of the at least one vascular access device, any combination thereof, and/or the like.

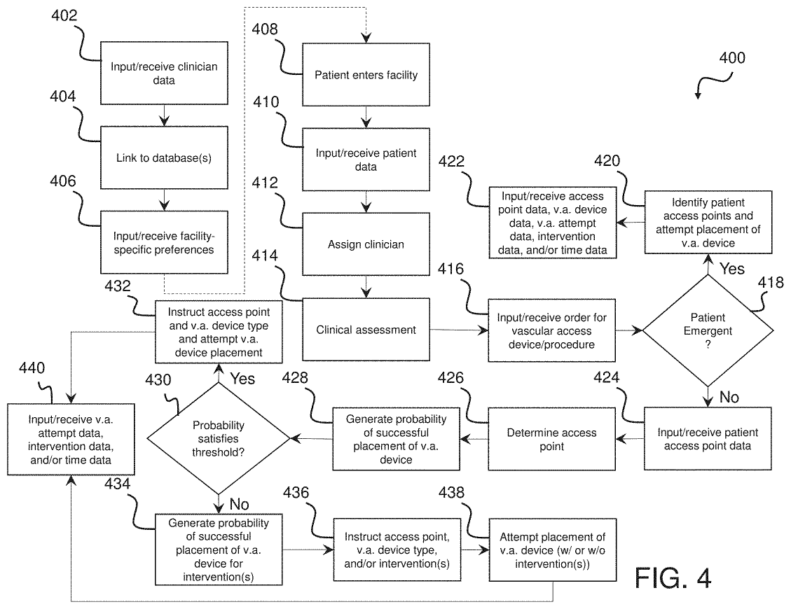

Referring now to FIG. 4, FIG. 4 is a diagram of an exemplary implementation 400 of a non-limiting embodiment relating to process 300 shown in FIG. 3. In some non-limiting embodiments or aspects, one or more of the steps of implementation 400 may be performed (e.g., completely, partially, and/or the like) by vascular access device placement modeling system 130 (e.g., one or more devices of vascular access device placement modeling system 130). In some non-limiting embodiments or aspects, one or more of the steps of implementation 400 may be performed (e.g., completely, partially, and/or the like) by another system, another device, another group of systems, or another group of devices, separate from or including vascular access device placement modeling system 130, such as medical device 102, medical device identification device 104, clinician identification device 110, patient identification device 112, reader device 120, database 132, user device 140, and/or the like. In some non-limiting embodiments, a vascular access device may be the same as or similar to medical device 102.

As shown in FIG. 4, at step 402, implementation 400 may include receiving clinician data (e.g., associated with a plurality of clinicians), as described herein. For example, vascular access device placement modeling system 130 may receive (e.g., retrieve) clinician data from database 132, as described herein. Additionally or alternatively, at least some of the clinician data may be inputted (e.g., via user device 140), and such data may be communicated to vascular access device placement modeling system 130 and/or database 132, as described herein.

As shown in FIG. 4, at step 404, implementation 400 may include linking vascular access device placement modeling system 130 to at least one database 132, as described herein. For example, vascular access device placement modeling system 130 may be connected to network 150 and/or directly connected to database(s) 132. Additionally or alternatively, database(s) 132 may be connected to network 150 and/or directly connected to vascular access device placement modeling system 130. In some non-limiting embodiments, vascular access device placement modeling system 130 may be granted access to database(s) 132 and/or data stored thereon.

As shown in FIG. 4, at step 406, implementation 400 may include receiving preferences data (e.g., facility-specific preferences data). For example, vascular access device placement modeling system 130 may receive (e.g., retrieve) preferences data from database 132, as described herein. Additionally or alternatively, at least some of the preferences data may be inputted (e.g., via user device 140), and such data may be communicated to vascular access device placement modeling system 130 and/or database 132, as described herein.

As shown in FIG. 4, at step 408, implementation 400 may include a patient (e.g., a first patient) entering a facility (e.g., hospital, ward, and/or the like). For example, the patient may be provided (a first) patient identification device 112.

As shown in FIG. 4, at step 410, implementation 400 may include receiving patient data. For example, vascular access device placement modeling system 130 may receive (e.g., retrieve) patient data from database 132 (e.g., based on patient identification data received from patient identification device 112 via reader device 120), as described herein. Additionally or alternatively, at least some of the patient data may be inputted (e.g., via user device 140), and such data may be communicated to vascular access device placement modeling system 130 and/or database 132, as described herein.

As shown in FIG. 4, at step 412, implementation 400 may include assigning a clinician (e.g., first clinician) to the patient. For example, vascular access device placement modeling system 130 may automatically assign the first clinician to the first patient (e.g., based on scheduling data associated with a schedule for each clinician of the plurality of clinicians). Additionally or alternatively, vascular access device placement modeling system 130 may receive (first) clinician identification data from (a first) clinician identification device 110 (e.g., via reader device 120) and/or may automatically determine that the first clinician was assigned to the first patient based on the reception of the first clinician identification data. Additionally or alternatively, the first clinician identification data may be inputted (e.g., via user device 140), and such data may be communicated to vascular access device placement modeling system 130 and/or database 132 to indicate that the first clinician was assigned to the first patient.

As shown in FIG. 4, at step 414, implementation 400 may include clinically assessing the (first) patient. For example, the first clinician may perform a clinical assessment of the first patient.

As shown in FIG. 4, at step 416, implementation 400 may include receiving an order for placement of a vascular access device for the first patient. For example, the first clinician may determine whether to order the placement of the vascular access device for the first patient based on the clinical assessment. Additionally or alternatively, the first clinician may input order data associated with the order for placement of a vascular access device for the first patient (e.g., via user device 140), and such data may be communicated to vascular access device placement modeling system 130 and/or database 132.

As shown in FIG. 4, at step 418, implementation 400 may include determining whether the first patient is emergent. For example, the first clinician may determine whether the first patient is emergent based on the clinical assessment. Additionally or alternatively, the first clinician may input emergent data associated with the first patient being emergent (e.g., via user device 140), and such data may be communicated to vascular access device placement modeling system 130 and/or database 132.

If the first patient is determined to be emergent, as shown in FIG. 4, at step 420, implementation 400 may include identifying access points of the patient and/or attempting to place a vascular access device. In some non-limiting embodiments or aspects, vascular access device placement modeling system 130 may receive (e.g., retrieve) access point data, vascular access device data, guideline data, cost data, preferences data, any combination thereof, and/or the like from database 132, as described herein. Additionally or alternatively, vascular access device placement modeling system 130 may generate an instruction message indicating an instruction to place a first vascular access device at a first access point based on the guideline data, the vascular access device data, the cost data, the preferences data, any combination thereof, and/or the like, as described herein, and the instruction message may be communicated (e.g., to user device 140 and/or the like), as described herein. In some non-limiting embodiments or aspects, the first clinician may manually identify at least one access point of the patient (e.g., based on and/or as part of the clinical assessment). In some non-limiting embodiments or aspects, the first clinician may attempt to place a vascular access device (e.g., first vascular access device) at the identified access point of the patient.

As shown in FIG. 4, at step 422, implementation 400 may include receiving data associated with the attempted placement of the vascular access device, as described herein. For example, vascular access device placement modeling system 130 may receive access point data, vascular access device data, vascular access attempt data, intervention data, time data, any combination thereof, and/or the like, as described herein. Additionally or alternatively, at least some of the data associated with the attempted placement of the vascular access device may be inputted (e.g., via user device 140), and such data may be communicated to vascular access device placement modeling system 130 and/or database 132, as described herein.

If the first patient is not determined to be emergent, as shown in FIG. 4, at step 424, implementation 400 may include receiving access point data, as described herein. For example, vascular access device placement modeling system 130 may receive access point data of the patient (e.g., retrieve at least some of the access point data from database 132 and/or the like), as described herein. Additionally or alternatively, at least some of the access point data may be inputted (e.g., via user device 140), and such data may be communicated to vascular access device placement modeling system 130 and/or database 132, as described herein.

As shown in FIG. 4, at step 426, implementation 400 may include determining an access point (e.g., first access point of the plurality of access points), as described herein. For example, vascular access device placement modeling system 130 may receive (e.g., retrieve) access point data, vascular access device data, guideline data, cost data, preferences data, any combination thereof, and/or the like from database 132, as described herein. Additionally or alternatively, vascular access device placement modeling system 130 may determine a first access point based on the guideline data, the vascular access device data, the cost data, the preferences data, any combination thereof, and/or the like, as described herein. For example, the first access point (e.g., target vein and/or the like) for a particular vascular access procedure may be determined based on long term outcomes associated with the first access point (e.g., based on access point data, guideline data, and/or the like), long term need to maintain access at the first access point (e.g., based on access point data, guideline data, and/or the like), preferences for the facility (e.g., based on preferences data, access point data, vascular access device data, guideline data, cost data, any combination thereof, and/or the like), any combination thereof, and/or the like. For the purpose of illustration, preferences data may indicate a preference for more distal access points if such access points are sufficient for long term outcomes and long term need to maintain access.

As shown in FIG. 4, at step 428, implementation 400 may include generating at least one first probability of successful placement of at least one vascular access device by the first clinician at the first access point of the first patient, as described herein. For example, vascular access device placement modeling system 130 may generate at least one first probability of successful placement of at least one vascular access device by the first clinician at the first access point of the first patient based on the patient data associated with the first patient, the clinician data associated with the first clinician, the vascular access device data associated with each of the at least one vascular access device, access point data associated with the first access point, any combination thereof, and/or the like. In some non-limiting embodiments, the first probability of successful placement of the vascular access device may include a probability of successful placement of the vascular access device on a first attempt to place the vascular access device (e.g., first stick).

As shown in FIG. 4, at step 430, implementation 400 may include determining whether the at least one first probability satisfies a threshold, as described herein. For example, vascular access device placement modeling system 130 may determine whether each first probability satisfies the threshold. In some non-limiting embodiments or aspects, the threshold may be determined based on (e.g., set based on and/or the like) preferences data, as described herein.

If the at least one first probability satisfies the threshold, as shown in FIG. 4, at step 432, implementation 400 may include instructing a first access point for attempting to place a first vascular access device and/or attempting to place the first vascular access device, as described herein. For example, vascular access device placement modeling system 130 may generate an instruction message indicating an instruction to place a first vascular access device at a first access point based on the first probability, the guideline data, the vascular access device data, the cost data, the preferences data, any combination thereof, and/or the like, as described herein, and/or the instruction message may be communicated (e.g., to user device 140 and/or the like), as described herein. Additionally or alternatively, the first clinician may attempt to place the first vascular access device (e.g., first type of vascular access device) at the first access point of the patient.

If the at least one first probability does not satisfy the threshold, as shown in FIG. 4, at step 434, implementation 400 may include generating at least one additional probability of successful placement of at least one vascular access device using at least one intervention, as described herein. For example, vascular access device placement modeling system 130 may generate at least one additional probability of successful placement of at least one vascular access device using the at least one intervention, as described herein. In some non-limiting embodiments or aspects, vascular access device placement modeling system 130 may generate at least one second probability of successful placement of the at least one vascular access device by the first clinician at the first access point of the first patient based on the patient data associated with the first patient, the clinician data associated with the first clinician, guidance data associated with at least one guidance system, any combination thereof, and/or the like. Additionally or alternatively, vascular access device placement modeling system 130 may generate at least one third probability of successful placement of the at least one vascular access device by a second clinician at the first access point of the first patient based on the patient data associated with the first patient, the clinician data associated with the second clinician, any combination thereof, and/or the like. Additionally or alternatively, vascular access device placement modeling system 130 may generate at least one fourth probability of successful placement of the at least one vascular access device by the second clinician at the first access point of the first patient based on the patient data associated with the first patient, the clinician data associated with the second clinician, the guidance data associated with at least one guidance system, any combination thereof, and/or the like. In some non-limiting embodiments, each probability (e.g., second probability, third probability, fourth probability, and/or the like) of successful placement of the vascular access device may include a probability of successful placement of the vascular access device on a first attempt to place the vascular access device (e.g., first stick).

As shown in FIG. 4, at step 436, implementation 400 may include instructing a first access point for attempting to place a first vascular access device and/or at least one intervention for such attempted placement, as described herein. For example, vascular access device placement modeling system 130 may generate an instruction message indicating an instruction to place a first vascular access device at a first access point using at least one intervention (e.g., second clinician, guidance system, any combination thereof, and/or the like) based on the first probability, the at least on additional probability (e.g., second probability, third probability, fourth probability, and/or the like), the guideline data, the vascular access device data, the cost data, the preferences data, any combination thereof, and/or the like, as described herein, and/or the instruction message may be communicated (e.g., to user device 140 and/or the like), as described herein. Additionally or alternatively, vascular access device placement modeling system 130 may generate an instruction message indicating an instruction to place a first vascular access device at a first access point by the first clinician (e.g., without any of the interventions, e.g., because the at least one additional probability was not sufficiently higher than the first probability) based on the first probability, the at least one additional probability (e.g., second probability, third probability, fourth probability, and/or the like), the guideline data, the vascular access device data, the cost data, the preferences data, any combination thereof, and/or the like, as described herein, and/or the instruction message may be communicated (e.g., to user device 140 and/or the like), as described herein.

As shown in FIG. 4, at step 438, implementation 400 may include attempting to place the first vascular access device (e.g., first type of vascular access device) at the first access point of the patient. For example, the first clinician and/or second clinician may attempt to place the first vascular access device (e.g., first type of vascular access device) at the first access point of the patient based on the instruction message, as described herein.

As shown in FIG. 4, at step 440, implementation 400 may include receiving data associated with the attempted placement of the vascular access device, as described herein. For example, vascular access device placement modeling system 130 may receive access point data, vascular access device data, vascular access attempt data, intervention data, time data, any combination thereof, and/or the like, as described herein. Additionally or alternatively, at least some of the data associated with the attempted placement of the vascular access device may be inputted (e.g., via user device 140), and such data may be communicated to vascular access device placement modeling system 130 and/or database 132, as described herein.

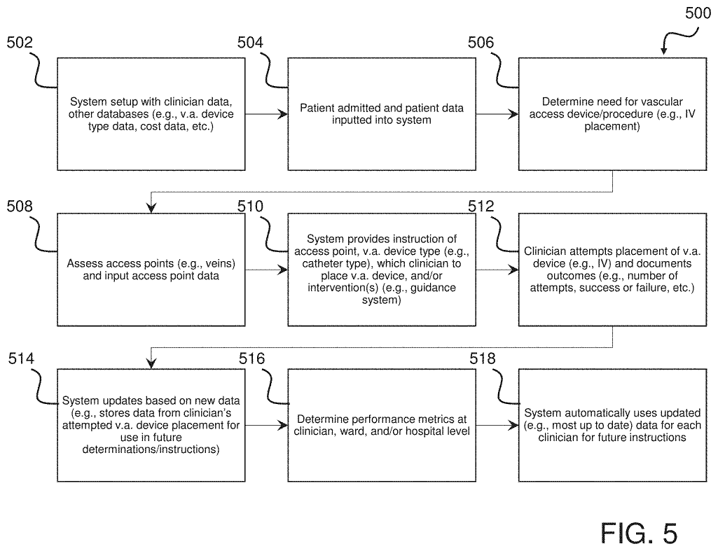

Referring now to FIG. 5, FIG. 5 is a diagram of an exemplary implementation 500 of a non-limiting embodiment relating to process 300 shown in FIG. 3. In some non-limiting embodiments or aspects, one or more of the steps of implementation 500 may be performed (e.g., completely, partially, and/or the like) by vascular access device placement modeling system 130 (e.g., one or more devices of vascular access device placement modeling system 130). In some non-limiting embodiments or aspects, one or more of the steps of implementation 500 may be performed (e.g., completely, partially, and/or the like) by another system, another device, another group of systems, or another group of devices, separate from or including vascular access device placement modeling system 130, such as medical device 102, medical device identification device 104, clinician identification device 110, patient identification device 112, reader device 120, database 132, user device 140, and/or the like. In some non-limiting embodiments, a vascular access device may be the same as or similar to medical device 102.

As shown in FIG. 5, at step 502, implementation 500 may include setting up vascular access device placement modeling system 130 and/or database(s) 132. For example, vascular access device placement modeling system 130 and/or database(s) 132 may receive (e.g., retrieve, receive as input from user device 140, and/or the like) clinician data, as described herein. Additionally or alternatively, vascular access device placement modeling system 130 and/or database(s) 132 may receive (e.g., retrieve, receive as input from user device 140, and/or the like) other data such as patient data, preferences data, scheduling data, clinician identification data, patient identification data, vascular access device identification data, access point data, vascular access device data, guideline data, cost data, vascular access attempt data (e.g., historical vascular access attempt data), intervention data, guidance data, time data, any combination thereof, and/or the like, as described herein. In some non-limiting embodiments, vascular access device placement modeling system 130 may be connected to network 150 and/or directly connected to database(s) 132. Additionally or alternatively, database(s) 132 may be connected to network 150 and/or directly connected to vascular access device placement modeling system 130. In some non-limiting embodiments, vascular access device placement modeling system 130 may be granted access to database(s) 132 and/or data stored thereon.

As shown in FIG. 5, at step 504, implementation 500 may include receiving patient data. For example, a first patient may enter and/or be admitted into a facility. Additionally or alternatively, vascular access device placement modeling system 130 may receive (e.g., retrieve) patient data from database 132 (e.g., based on patient identification data received from patient identification device 112 via reader device 120), as described herein. Additionally or alternatively, at least some of the patient data may be inputted (e.g., via user device 140), and such data may be communicated to vascular access device placement modeling system 130 and/or database 132, as described herein. In some non-limiting embodiments or aspects, the (first) patient may be provided (a first) patient identification device 112.

As shown in FIG. 5, at step 506, implementation 500 may include determining that the first patient needs a vascular access device and/or vascular access procedure. For example, a first clinician may be assigned to the first patient, and/or the first clinician may perform a clinical assessment of the first patient. Additionally or alternatively, the first clinician may determine whether to order the placement of the vascular access device for the first patient based on the clinical assessment. In some non-limiting embodiments or aspects, the first clinician may input order data associated with the order for placement of a vascular access device for the first patient (e.g., via user device 140), and such data may be communicated to vascular access device placement modeling system 130 and/or database 132.

As shown in FIG. 5, at step 508, implementation 500 may include receiving access point data, as described herein. For example, vascular access device placement modeling system 130 may receive access point data of the patient (e.g., retrieve at least some of the access point data from database 132 and/or the like), as described herein. Additionally or alternatively, at least some of the access point data may be inputted (e.g., via user device 140), and such data may be communicated to vascular access device placement modeling system 130 and/or database 132, as described herein. For example, the first clinician may assess at least some access points of the first patient and input access point data associated therewith into user device 140, which may communicate such data to vascular access device placement modeling system 130 and/or database 132, as described herein.

As shown in FIG. 5, at step 510, implementation 500 may include providing (e.g., generating and/or communicating) an instruction message indicating an instruction to place a first vascular access device at a first access point of the first patient. For example, vascular access device placement modeling system 130 may generate at least one first probability of successful placement of at least one vascular access device by the first clinician at the first access point of the first patient based on the patient data associated with the first patient, the clinician data associated with the first clinician, and/or the like, as described herein. Additionally or alternatively, vascular access device placement modeling system 130 may generate at least one additional probability of successful placement of at least one vascular access device using the at least one intervention, as described herein. In some non-limiting embodiments, vascular access device placement modeling system 130 may generate an instruction message indicating an instruction to place a first vascular access device at a first access point based on the first probability, the guideline data, the vascular access device data, the cost data, the preferences data, any combination thereof, and/or the like, as described herein. Additionally or alternatively, vascular access device placement modeling system 130 may generate an instruction message indicating an instruction to place a first vascular access device at a first access point using at least one intervention (e.g., second clinician, guidance system, any combination thereof, and/or the like) based on the first probability, the at least one additional probability (e.g., second probability, third probability, fourth probability, and/or the like), the guideline data, the vascular access device data, the cost data, the preferences data, any combination thereof, and/or the like, as described herein. In some non-limiting embodiments, vascular access device placement modeling system 130 may communicate the instruction message (e.g., to user device 140 and/or the like), as described herein.

As shown in FIG. 5, at step 512, implementation 500 may include attempting to place the first vascular access device (e.g., first type of vascular access device) at the first access point of the patient. For example, the first clinician and/or second clinician may attempt to place the first vascular access device (e.g., first type of vascular access device) at the first access point of the patient based on the instruction message, as described herein. In some non-limiting embodiments or aspects, the outcome(s) of the attempted placement of the vascular access device may be documented. For example, vascular access device placement modeling system 130 may receive data associated with the attempted placement of the vascular access device, as described herein. For example, vascular access device placement modeling system 130 may receive access point data, vascular access device data, vascular access attempt data, intervention data, time data, any combination thereof, and/or the like, as described herein. Additionally or alternatively, at least some of the data associated with the attempted placement of the vascular access device may be inputted (e.g., by the first clinician and/or second clinician via user device 140), and such data may be communicated to vascular access device placement modeling system 130 and/or database 132, as described herein.

As shown in FIG. 5, at step 514, implementation 500 may include updating based on the received data associated with the attempted placement of the vascular access device. For example, vascular access device placement modeling system 130 and/or database 132 may store such data, e.g., for use in future determinations (e.g., generation of the probabilities, as described herein) and/or for use in future instructions (e.g., generation of the instruction messages, as described herein).

As shown in FIG. 5, at step 516, implementation 500 may include determining performance metrics. For example, vascular access device placement modeling system 130 may determine performance metrics for at least one of a clinician, a facility (e.g., ward, hospital, building, and/or the like), any combination thereof, and/or the like, as described herein.

As shown in FIG. 5, at step 518, implementation 500 may include using the updated data (e.g., most recent/up-to-date, as stored by vascular access device placement modeling system 130 and/or database 132) for future instructions. For example, vascular access device placement modeling system 130 may use the updated data for at least one future determination (e.g., generation of at least one probability of successful placement of a vascular access device at an access point of a patient, as described herein) and/or for generating at least one instruction message (e.g., generation an instruction message for vascular access device placement modeling system 130 and/or database 132, as described herein).

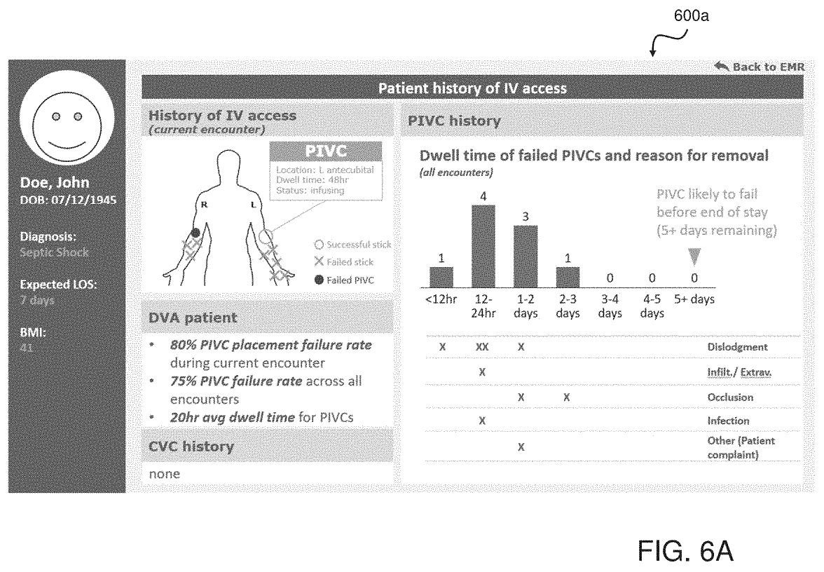

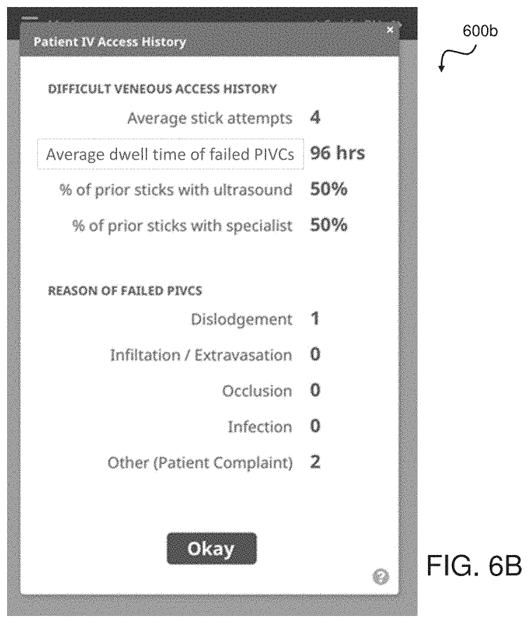

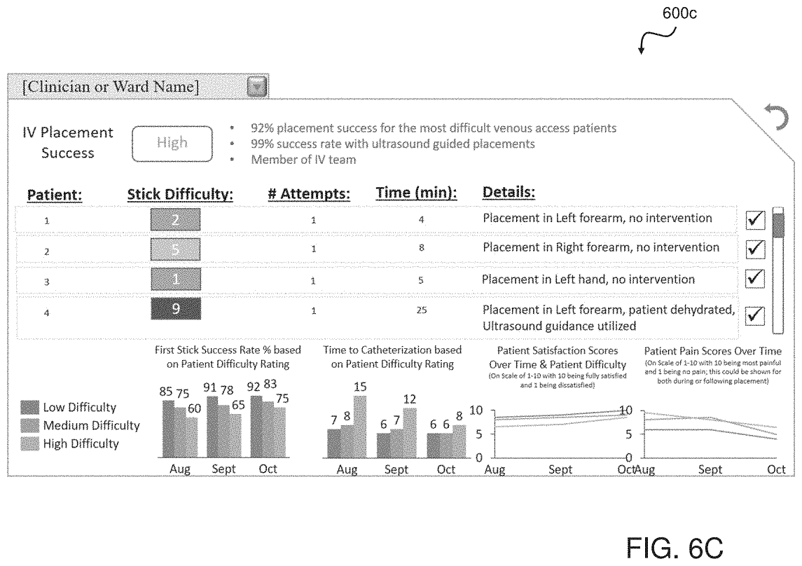

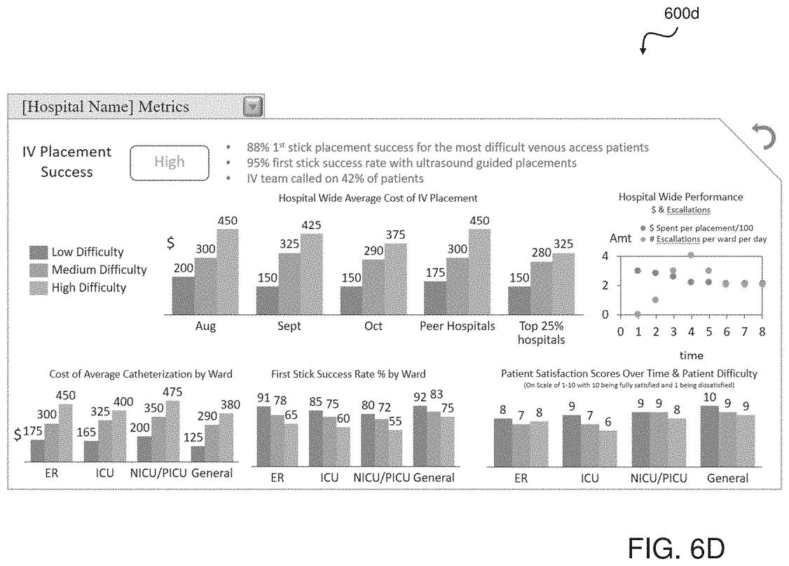

Referring now to FIGS. 6A-6D, FIGS. 6A-6D are screenshots of exemplary graphical user interfaces 600a-600d of non-limiting embodiments relating to process 300 shown in FIG. 3. In some non-limiting embodiments or aspects, one or more of the graphical user interfaces 600a-600d may be displayed (e.g., completely, partially, and/or the like) by user device 140. In some non-limiting embodiments or aspects, one or more of the graphical user interfaces 600a-600d may be displayed (e.g., completely, partially, and/or the like) by another system, another device, another group of systems, or another group of devices, separate from or including user device 140, such as reader device 120, vascular access device placement modeling system 130, database 132, and/or the like.

As shown in FIG. 6A, in some non-limiting embodiments or aspects, graphical user interface 600a may include patient identification data, such as an image of the patient's face, the patient's name, the patient's birthdate, any combination thereof, and/or the like. In some non-limiting embodiments or aspects, graphical user interface 600a may include diagnosis data for the patient, expected length of stay (LOS) data for the patient, BMI data for the patient, any combination thereof, and/or the like. In some non-limiting embodiments or aspects, graphical user interface 600a may include other patient data (e.g., historical patient data), such as a history of attempted placement of vascular access devices for the current encounter with the patient (e.g., “History of IV access (current encounter)”), Difficult Intra Venous Access (DIVA) and/or Difficult Venous Access (DVA) data for the patient (e.g., “DVA patient”), central venous catheter (CVC) history for the patient (“CVC history”), peripheral intravenous catheters (PIVC) history for the patient (“PIVC history”), any combination thereof, and/or the like. For example, the history of attempted placement of vascular access devices for the current encounter with the patient may include a diagram showing failed attempts at placement of a vascular access device (e.g., “Failed stick” denoted by a first icon (e.g., an X) having a first color), failed vascular access devices (e.g., “Failed PIVC” denoted by a second icon (e.g., a filled circle) having a second color), and successful placements of at least one vascular access device (e.g., “Successful stick” denoted by a third icon (e.g., a non-filled circle) having a third color). Additionally or alternatively, the DVA data for the patient may include a failure rate of vascular access devices (e.g., PIVCs and/or the like) during a current encounter, a failure rate of vascular access devices during a patient's entire medical history (e.g., across all encounters), an average dwell time for each vascular access device, any combination thereof, and/or the like. Additionally or alternatively, the CVC history for the patient may include an indication whether the patient currently has and/or has had a CVC or not. Additionally or alternatively, the PIVC history for the patient may include at least one chart indicating the dwell time for each vascular access device (e.g., PIVC and/or the like) and/or the reason for removal thereof.

As shown in FIG. 6B, graphical user interface 600b may include patient data, e.g., historical data associated with attempted placements of vascular access devices for the patient (e.g., “Patient IV Access History”). For example, the patient data may include a DVA history (“DIFFICULT VENOUS ACCESS HISTORY”), data associated with reasons for failed vascular access devices (e.g., “REASONS OF FAILED PIVCS”), any combination thereof, and/or the like. In some non-limiting embodiments or aspects, the DVA history may include an average number of attempted placements of vascular access devices per encounter with the patient (e.g., “Average stick attempts”), an average dwell time for each failed vascular access device (e.g., “Average dwell time of failed PIVCs”), a percentage of attempted placements of vascular access devices using at least one guidance system (e.g., “% of prior sticks with ultrasound”), a percentage of attempted placements of vascular access devices using a specialist such as a member of an IV team or an experienced clinician (e.g., “% of prior sticks with specialist”), any combination thereof, and/or the like. Additionally or alternatively, the data associated with reasons for failed vascular access devices may indicate a number of such vascular access devices that failed for each possible reason for failure (e.g., dislodgment, infiltration/extravasation, occlusion, infection, other (e.g., patient complaint), any combination thereof, and/or the like).

As shown in FIG. 6C, graphical user interface 600c may include performance metrics for at least one of a clinician, a facility (e.g., ward and/or the like), any combination thereof, and/or the like, as described herein.

As shown in FIG. 6D, graphical user interface 600d may include performance metrics for at least one facility (e.g., hospital and/or the like) and/or the like, as described herein.

Although the disclosed subject matter has been described in detail for the purpose of illustration based on what is currently considered to be the most practical and preferred embodiments or aspects, it is to be understood that such detail is solely for that purpose and that the disclosed subject matter is not limited to the disclosed embodiments or aspects, but, on the contrary, is intended to cover modifications and equivalent arrangements that are within the spirit and scope of the appended claims. For example, it is to be understood that the presently disclosed subject matter contemplates that, to the extent possible, one or more features of any embodiment or aspect can be combined with one or more features of any other embodiment.

What is claimed is:

1. A method for vascular access device placement, comprising:
receiving, with at least one processor, clinician data associated with at least one clinician, the at least one clinician comprising a first clinician;
receiving, with at least one processor, patient data associated with at least one patient, the at least one patient comprising a first patient;
generating, with at least one processor, at least one first probability of successful placement of at least one vascular access device by the first clinician at at least one access point of the first patient based on the patient data associated with the first patient and the clinician data associated with the first clinician; and
communicating, with at least one processor, an instruction message for placement of the at least one vascular access device based on the at least one first probability to a device user.

2. The method of claim 1, wherein the clinician data comprises at least one of years of experience, handedness, level of training, years of degree, whether the clinician is a member of an intravenous (IV) team, salary, a number of attempted placements of vascular access devices, a number of successful placements of the vascular access devices, a number of attempts per successful placement of each vascular access device, a number of attempted placements of the vascular access devices per patient, a vascular access device placement success ratio, an access point for each attempted placement of the vascular access devices, a number of successful placements of the vascular access devices per selectable subperiod over a selectable time period, whether at least one guidance system was used for each attempted placement of the vascular access devices, whether the at least one guidance system was used for each successful placement of the vascular access devices, or any combination thereof.

3. The method of claim 1, wherein the patient data comprises at least one of height, weight, body mass index (BMI), sex, handedness, dominant side, current diagnostic code, one or more comorbidity diagnostic codes, previous intravenous (IV) treatments, skin pallor, skin shade, whether the patient has a tremor or other involuntary movement disorder, whether the patient has a condition requiring restraint or sedation, a categorization as Difficult Intra Venous Access (DIVA) or Adult Difficult Intra Venous Access (A-DIVA), an American Society of Anesthesiologists (ASA) physical status classification, whether the patient has an unplanned indication for surgery, whether the patient has undergone preoperative fasting, palpability of a target vein at the at least one access point, visibility of a target vein at the at least one access point, a known history of difficult peripheral IV cannulation, a dilated vein diameter smaller than a selectable threshold, size of the vascular access device, at least one available access point on at least one extremity of the patient, whether the patient has at least one vascular disease, whether the patient has preoperative hypovolemia, whether the patient has renal insufficiency, a level of fear of needles of the patient, a measure of difficulty of maintaining peripheral IV access once a respective vascular access device is placed, a number of attempted placements of vascular access devices for the patient, a number of successful placements of the vascular access devices for the patient, a number of attempts per successful placement of each vascular access device for the patient, a vascular access device placement success ratio for the patient, an access point for each attempted placement of the vascular access devices for the patient, or any combination thereof.

4. The method of claim 1, wherein generating the at least one first probability comprises generating the at least one first probability using a neural network model, the neural network model comprising at least one neural network trained based on historical clinician data associated with a plurality of clinicians and historical patient data associated with a plurality of patients.

5. The method of claim 4, further comprising, before generating the at least one first probability, training, with at least one processor, the at least one neural network of the neural network model based on the historical clinician data and the historical patient data.

6. The method of claim 1, further comprising:
receiving, with at least one processor, first patient access point data associated with the at least one access point of the first patient,
wherein generating the at least one first probability comprises generating the at least one first probability based on the patient data associated with the first patient, the clinician data associated with the first clinician, and the first patient access point data.

7. The method of claim 1, wherein the at least one clinician comprises a plurality of clinicians comprising the first clinician, wherein the at least one patient comprises a plurality of patients comprising the first patient, the method further comprising:
receiving, with at least one processor, first clinician identification data associated with the first clinician of the plurality of clinicians; and
receiving, with at least one processor, first patient identification data associated with the first patient of the plurality of patients.

8. The method of claim 1, further comprising:
receiving, with at least one processor, guideline data associated with at least one recommended access point for each of at least one vascular access procedure;
receiving, with at least one processor, vascular access device data associated with the at least one vascular access device and cost data associated with supplies required for an attempted placement of the at least one vascular access device; and
generating, with at least one processor, the instruction message indicating an instruction to place a first vascular access device of the at least one vascular access devices at a first access point of the at least one access point based on the at least one first probability, the guideline data, the vascular access device data, and the cost data.

9. The method of claim 1, further comprising, before communicating the instruction message:
determining, with at least one processor, that the at least one first probability satisfies a threshold; and
generating, with at least one processor, the instruction message indicating an instruction for the first clinician to place the at least one vascular access device at a first access point of the at least one access point based on the at least one first probability satisfying the threshold.

10. The method of claim 1, further comprising, before communicating the instruction message:
determining, with at least one processor, that the first probability does not satisfy a threshold; and
generating, with at least one processor, the instruction message indicating an instruction for at least one intervention for placement of the at least one vascular access device based on the at least one first probability failing to satisfy the threshold,
wherein the at least one intervention comprises at least one of a second clinician for placement of the vascular access device, at least one guidance system for placement of the vascular access device, or any combination thereof.

11. The method of claim 10, further comprising, before generating the instruction message:
generating at least one of:
at least one second probability of successful placement of the at least one vascular access device by the first clinician at the at least one access point of the first patient based on the patient data associated with the first patient, the clinician data associated with the first clinician, and guidance data associated with at least one guidance system;
at least one third probability of successful placement of the at least one vascular access device by a second clinician of the at least one clinician at the at least one access point of the first patient based on the patient data associated with the first patient and the clinician data associated with the second clinician; or
at least one fourth probability of successful placement of the at least one vascular access device by the second clinician at the at least one access point of the first patient based on the patient data associated with the first patient, the clinician data associated with the second clinician, and the guidance data associated with at least one guidance system.

12. The method of claim 1, further comprising:
generating, with at least one processor, at least one first predicted patient satisfaction for an attempted placement of the at least one vascular access device by the first clinician at the at least one access point of the first patient based on the patient data associated with the first patient and the clinician data associated with the first clinician; and
generating the instruction message for placement of the at least one vascular access device based on the at least one first probability and the at least one first predicted patient satisfaction,
wherein communicating the instruction message comprises communicating the instruction message for placement of the at least one vascular access device based on the at least one first probability and the at least one first predicted patient satisfaction to the user device.

13. The method of claim 1, further comprising:
receiving, with at least one processor, input data based on an attempted placement of the at least one vascular access device by the first clinician at the at least one access point of the first patient, the input data comprising at least one of:
an update to the patient data associated with the first patient;
an update to the clinician data associated with the first clinician;
a side of the patient in which the attempted placement of the at least one vascular access device was performed;
a first access point of the at least one access point at which the attempted placement of the at least one vascular access device was performed;
a visibility of a target vein at the at least one access point;
a dilated vein diameter of the target vein at the at least one access point;
a palpability of the target vein at the at least one access point;
a size of the at least one vascular access device;
a type of the at least one vascular access device;
a number of attempted placements of the at least one vascular access device;
an indication of whether the attempted placement of the at least one vascular access device was successful;
a time from when placement of the at least one vascular access device was ordered to the attempted placement of the at least one vascular access device;
an indication of whether at least one intervention for placement of the at least one vascular access device was used;
a level of fear of the attempted placement of the at least one vascular access device of the patient;
a first pain score of the patient during the attempted placement of the at least one vascular access device;
a second pain score of the patient after the attempted placement of the at least one vascular access device;
a patient satisfaction score for the attempted placement of the at least one vascular access device; or
any combination thereof.

14. The method of claim 1, further comprising:
generating, with at least one processor, at least one of:
at least one predicted cost for placement of the at least one vascular access device;
at least one expected time from when placement of the at least one vascular access device was ordered to the successful placement of the at least one vascular access device;
at least one first expected pain score of the patient during placement of the at least one vascular access device;
at least one second expected pain score of the patient after placement of the at least one vascular access device;
at least one difficulty score for placement of the at least one vascular access device at the at least one access point of the first patient; or
any combination thereof.

15. The method of claim 1, further comprising:
generating, with at least one processor, at least one graphical user interface (GUI) indicating at least one of:
an average time from when placement of the at least one vascular access device was ordered to the successful placement of the at least one vascular access device as a function of patient difficulty;
a vascular access device placement success ratio as a function of the patient difficulty;
a first average pain score during placement of the at least one vascular access device as a function of patient difficulty;
a second average pain score after placement of the at least one vascular access device as a function of patient difficulty;
an average patient satisfaction score;
a summary of placements of vascular access devices; or
any combination thereof.

16. A system comprising:
at least one clinician identification device, the at least one clinician identification device comprising a first clinician identification device associated with a first clinician;
at least one patient identification device, the at least one patient identification device comprising a first patient identification device associated with a first patient;
at least one vascular access device, each vascular access device comprising a vascular access device identification device associated therewith;
at least one reader device configured to communicate with the at least one clinician identification device, the at least one patient identification device, and each vascular access device identification device, the reader connected to a communication network;
at least one user device connected to the communication network;
at least one database connected to the communication network, the at least one database configured to:
store clinician data associated with a plurality of clinicians, the plurality of clinicians comprising the first clinician;
store patient data associated with a plurality of patients, the plurality of patients comprising the first patient, the patient data for the first patient comprising access point data associated with at least one access point of the first patient; and
store vascular access device data associated with a plurality of vascular access devices, the plurality of vascular access devices comprising the at least one vascular access device; and
a vascular access device placement modeling system connected to the communication network, the vascular access device placement modeling system configured to:
receive first clinician identification data associated with the first clinician from the first clinician identification device via the reader device;
receive first patient identification data associated with the first patient from the first patient identification device via the reader device;
receive vascular access device identification data associated with the at least one vascular access device from the vascular access device identification device associated with each of the at least one vascular access device via the reader device;
retrieve the clinician data associated with the first clinician from the at least one database;
retrieve the patient data associated with the first patient from the at least one database;
retrieve the vascular access device data associated with each of the at least one vascular access device from the at least one database;
generate at least one first probability of successful placement of the at least one vascular access device by the first clinician at the at least one access point of the first patient based on the patient data associated with the first patient, the clinician data associated with the first clinician, and the vascular access device data associated with each of the at least one vascular access device; and
communicate an instruction message for placement of the at least one vascular access device based on the at least one first probability to the at least one user device.

17. The system of claim 16, wherein the at least one user device comprises the reader device.

18. The system of claim 16, wherein at least one server comprises the at least one database and the vascular access device placement modeling system.

19. The system of claim 16, wherein the at least one user device is configured to:
receive as input at least one of the clinician data, the patient data, the vascular access device data, or any combination thereof; and
communicate the input to the at least one database.

20. A computer program product for vascular access device placement comprising at least one non-transitory computer-readable medium including one or more instructions that, when executed by at least one processor, cause the at least one processor to:
receive clinician data associated with at least one clinician, the at least one clinician comprising a first clinician;
receive patient data associated with at least one patient, the at least one patient comprising a first patient;
generate at least one first probability of successful placement of at least one vascular access device by the first clinician at at least one access point of the first patient based on the patient data associated with the first patient and the clinician data associated with the first clinician; and
communicate an instruction message for placement of the at least one vascular access device based on the at least one first probability to a user device.
